(19) United States
(12) Patent Application Publication
(10) Pub. No.: US 2025/0228696 A1
(43) Pub. Date: Jul. 17, 2025

(54) ENDOLUMINAL STOMA DEVICE

(71) Applicant: Armstrong Medical, Inc., Atlanta, GA (US)

(72) Inventor: David N. Armstrong, Atlanta, GA (US)

(21) Appl. No.: 18/703,254

(22) PCT Filed: Oct. 21, 2022
(86) PCT No.: PCT/US2022/047461

§ 371 Date: Apr. 19, 2024

Related U.S. Application Data
(60) Provisional application No. 63/270,863, filed on Oct. 22, 2021.

(51) Int. Cl. A61F 5/448; A61F 5/44; A61F 5/441; A61F 5/445

(52) U.S. Cl. CPC A61F 5/448; A61F 5/4405; A61F 5/441; A61F 2005/4455; A61F 2005/4486

(57) ABSTRACT

A waste collection system for collecting waste from a body of a patient. The waste collection system includes: an endoluminal stoma channel having a resilient deformable inner ring, an outer ring, a flexible polymer sleeve made from a biocompatible polymer and interconnecting the inner ring and the outer ring; an inner colostomy bag which can be detachably attached to the outer ring to collect and contain the waste; and a cover which can engage the outer ring in a sealed engagement, and where a portion of the flexible polymer sleeve and the inner ring are configured for insertion into a stoma formed in the body of the patient.

### CROSS REFERENCE TO RELATED APPLICATIONS

This application claims priority to U.S. Provisional Patent Application Ser. No. 63/270,863 filed Oct. 22, 2021. The noted application is incorporated herein by reference.

### BACKGROUND

Colostomy plates typically consist of a square adhesive plate usually made of corya gum which is placed on the skin of the patient. The colostomy or the ileostomy protrudes through a central hole through the center of the plate and the colostomy bag is attached to/detached from the plate using a clip-on plastic device similar to a Zip-Lock bag. Colostomy plates are difficult to attach and remain attached to the skin of the patient especially if the skin is moist or if the skin becomes infected or irritated from the spillage of the stool. At this point, adhesives or glues are used in an attempt to stick the plate to the skin. Frequently, the plates fall off or the bag falls off and stool leaks onto the abdominal wall causing inconvenience and distress.

### SUMMARY OF INVENTION

This application extends the work described in PCT/US2021/34619 to Armstrong, filed May 27, 2021, entitled “Endoluinal Stoma Devices,” which claims priority to U.S. provisional patent application 63/031,285 filed May 28, 2020 to Armstrong, entitled “Endoluminal Stoma Devices,” both of which are incorporated herein by reference.

The current device avoids the need for colostomy plates, glues and adhesives. A self-retaining endoluminal colostomy device is provided which avoids the traditional stoma plates and adhesives. The device incorporates a flexible or semi-rigid plastic sleeve which is generally cylindrical in shape. A ring, which can be made of plastic or polymer or other springy material, is located at each end of the sleeve. The inner ring, which is generally deformable with return to or close to its original shape upon deformation and release, is inserted into the lumen of the stoma using an introducer or flexible endoscope. Once inserted, the inner ring unfolds and adopts its original circular form. By gently pulling on the outer ring, the inner ring is then pulled to the sub-fascial space where it becomes lodged. The outer ring can then be secured around the outer tip of the stoma to secure the channel through the device for ultimate collection of waste. In some embodiments, the outer ring is rolled over upon itself which shortens the plastic sleeve while securing the device around the stoma. The outer ring protects the stoma and forms a stable base to attach stoma devices such as colostomy bags or occlusive devices. The cylindrical sleeve, the inner ring and the outer ring therefore create a self-retaining device to attach stoma bags, occlusive plates, voiding tubes, or other suitable structures thereto. This design can avoid the use of plates, adhesives, or the like, and therefore avoids inflammation and/or ulceration of the peri-stomal skin.

The two rings may be equal or unequal in size and/or shape. There may also be a plurality of “inner rings”, to improve traction of the device within the bowel lumen. The inner ring may have the same diameter, or a slightly larger diameter than the diameter of the inner lumen of the small or the large bowel, so enabling it to become lodged in the sub-fascial space. The diameter of the outer ring may be slightly larger than the diameter of a conventional stoma, so therefore sized to provide for covering the external stoma. The sleeve membrane or a portion thereof may be fenestrated or contain perforations to allow mucous secretions into the lumen of the bowel.

In a first aspect, the invention pertains to an endoluminal stoma channel comprising a resilient deformable inner ring, an outer ring, a flexible polymer sleeve connected to the inner ring and the outer ring to form a flow channel and comprising a biocompatible polymer, and a tether comprising a cord and a knob. In general, the cord is attached to the inner ring, and the knob has a position such that the cord can extend along the sleeve, in the interior or exterior of the sleeve, past the outer ring with the outer ring pulled away from the inner ring such that the sleeve is taut. The words sleeve and membrane are used interchangeably herein for convenience.

In a further aspect, the invention pertains to a method for controlling release from a stoma through an endoluminal stoma channel comprising a resilient deformable inner ring, an outer ring, and a flexible polymer sleeve connected to the inner ring and the outer ring to form a flow channel and comprising a biocompatible polymer. The method comprises engaging an outer cover with a clamp onto the outer ring of the endoluminal stoma channel, wherein the inner ring is located within the patient with the flexible polymer sleeve extending outward from the stoma with the outer ring positioned outside the stoma, wherein the outer cover provides closure of the stoma or a controlled pathway for releases from the stoma.

In another aspect, the invention pertains to a stoma discharge control system comprising:
an endoluminal stoma channel comprising a resilient deformable inner ring, an outer ring, and a flexible polymer sleeve comprising a biocompatible polymer and connecting the inner ring and the outer ring; and
an outer cover comprising a clamp or seal element configured to engage the outer ring in a sealed engagement, wherein the outer cover provides an opening, a connector element, a closed surface or a combination thereof.

In yet another embodiment, the current device includes an outer flexible ring and an inner flexible ring interconnected by a thin transparent membrane. The inner ring is configured for insertion into a stoma (colostomy, ileostomy, urostomy) underneath the inner aspect of the abdominal wall of a patient. The outer ring is configured so that it may be folded inward or outward to shorten the plastic membrane making the inner ring snug underneath the abdominal wall. The outer ring may also take the form of various cross-sectional shapes to keep the ring from unfolding when folded inward or outward. The inner ring may also take the form of various cross-sectional shapes to enable the inner ring to be folded to adjust the length of the circular membrane, for instance shortened in a thin emaciated individual, or lengthened for instance in a morbidly obese individual. Once folded, the membrane can maintain its circular form as a result of the cross-sectional shape of the inner ring.

In a further embodiment the inner ring is made of a softer and more malleable material in order to minimize pressure on the underlying small bowel or large bowel mucosa and to prevent erosions or ischemia of the bowel wall.

In a still further embodiment, the inner ring contains circumferential protrusions, such as ribs, bumps, ridges, or other suitable shapes, and/or a textured surface in order to increase contact between the inner ring and the small or large bowel, to prevent the inner ring from slipping out of the stoma.

A third modification involves the circular membrane interconnecting the inner and outer ring being transparent in order to visualize the color of the stoma ensuring that it remains pink and viable and not dusky and ischemic. A further embodiment involves the dynamic nature of the circular membrane between the inner and outer rings, the circular membrane being operable to dampen any excessive intra-abdominal force such as during sudden coughing or sneezing (i.e., being operable to reversibly absorb, store, and return the energy used to deflect or distort the intra-abdominal force). This dynamic, elastic configuration acts in a similar way as a shock absorber. Any sudden increase in intra-abdominal pressure will put immediate pressure on the outer cap of the device. This will transiently increase tension in the plastic membrane interconnecting the inner and outer ring cushioning any sudden increase in pressure, in effect acting as a shock absorber between the inner and outer ring. This will reduce the chances of the inner ring being extruded.

Any increase in tension on the circular membrane will also increase pressure on the inner ring forcing it to become more securely positioned on the inner aspect of the stoma and the underside of the fascial surface of the anterior abdominal wall. This increased pressure will therefore increase the seal between the inner ring, the inner aspect of the stoma and the anterior abdominal wall, thereby preventing leaks of liquids around the inner ring and the outer surface of the inner membrane.

This dynamic system will act like a shock absorber for any sudden increase in the intra-abdominal pressure to prevent leaks from the device. A further aspect of the current invention involves the mechanical stability between the inner and outer rings provided by gentle traction on the circular membrane which gently squeezes the inner aspect of the stoma and the abdominal wall keeping the stoma device securely in place. This contrasts with the adynamic and rigid configuration of the cylindrical stoma device described in U.S. Pat. No. 4,137,918 to Bogert, which would be easily “shot” out of the stoma with a sudden increase in intra-abdominal pressure, such by a sudden cough or sneeze.

The current embodiment also includes the provision of an inner colostomy bag made of thin pliable plastic or latex, similar to a large condom or balloon. This inner colostomy bag is detachably attached to the outer ring using a snap-on mechanism, (similar to a Zip-Lock bag or conventional colostomy attachment) or a slip-on mechanism, in the case of the condom or balloon embodiment. Once attached to the outer ring, the inner colostomy bag is gently delivered into the inner aspect of the plastic membrane of the device, and the outer cover is attached to the outer ring securing the inner bag in the inner aspect of the stoma device.

When the patient feels the need to defecate, the outer cover is removed from the outer ring and the stool begins to fill the inner colostomy bag. As the inner colostomy bag fills, it's extruded to the outer aspect of the stoma device, similar to a conventional outer colostomy device. Once the colostomy bag is at least partially full it can be removed from the outer ring and the waste disposed of with minimal spillage of stool. The inner colostomy bag prevents the free spillage of stool from the stoma device once the outer cover is removed and permits clean and controlled delivery of stool into the bag which can then be disposed of cleanly.

In a further embodiment the inner colostomy bag may include a malleable colostomy ring positioned at a mouth (or opening) of the inner colostomy bag. The malleable colostomy ring is compressible and when compressed can be inserted inside the inner aspect of membrane below the outer ring. Upon release of compression, the colostomy ring returns to or close to its original shape and becomes sealingly engaged with the outer ring to prevent leakage of stool and gas. The inner colostomy bag may then be inserted in the inner aspect of the device and the outer cover attached to the outer ring. In some embodiments, the inner colostomy bag may further include a gas releasing mechanism. In one embodiment, the gas releasing mechanism includes an air release tube having an upper opening and a lower opening, the tube secured within a port in the inner colostomy bag such that the tube's lower opening is positioned in the inner aspect of the stoma device and the tube's upper opening is positioned in the outer aspect of the device. In one embodiment, the upper opening may be sealed or closed via a removable cap or by a valve and gas may be released through the tube's upper opening by removing the cap or opening the valve.

In yet a further embodiment, a tab or string may be positioned on the periphery of the colostomy ring to assist in removing or venting the inner colostomy bag and/or to release gas from the lumen of the bowel in the event of gas build-up. In some embodiments, the tab or string may further protrude through an opening on the outer cover when the outer cover is attached to the outer ring. Traction on the tab or string when the colostomy ring is sealingly engaged with the outer ring will cause the colostomy ring to deform thereby breaking the seal between the colostomy ring and the outer ring allowing for the removal or venting of the inner colostomy bag and/or release of gas from the lumen of the bowel. In a further embodiment, the tab or string may be positioned on the periphery of the colostomy ring and further detachably attached to the periphery of the cap and traction on the tab or string occurs by at least partially removing the cap from the outer ring. In one embodiment, the tab or string may be positioned on the periphery of the colostomy ring at a 12 o'clock position to preferentially allow for the escape of gas or flatus, rather than the escape of liquids or stool.

### BRIEF DESCRIPTION OF DRAWINGS

FIG. 1 is a front view of a conventional ostomy place positioned around a stoma.

FIG. 2 is a schematic sectional view depicting a section of bowel passing through an abdominal wall to form a stoma that has a conventional stoma plate attached around the stoma.

FIG. 3 is a perspective view of an embodiment of an endoluminal stoma channel, with various cross-sectional shapes of the inner and outer rings shown in the A1 to A7 and B1 to B7 sectional views taken along lines A-A and B-B.

FIG. 4 is a perspective view of an endoluminal stoma channel with the inner ring and outer ring being operable to be rolled over to shorten the sleeve.

FIG. 5 is a sectional side view depicting the dynamic relationship between the inner ring and the outer ring and the sleeve interconnecting the inner ring and outer ring.

FIG. 5A is a schematic sectional side view of a bowel connecting to a stoma passing through the abdominal wall with an endoluminal stoma channel positioned within the stoma.

FIG. 5B is a sectional side view of an embodiment where the inner ring of the endoluminal stoma channel is secured at a position closer to the exterior surface of the abdominal wall.

FIGS. 5C and 5D are sectional side views depicting a fluid-tight seal between the inner ring and the inner aspect of the stoma on the inner aspect of the abdominal wall during a sudden increase in intra-abdominal pressure.

FIG. 6A is a perspective view of an endoluminal stoma channel with an outer ring larger than the inner ring connected with a conical shaped sleeve.

FIG. 6B is a perspective view of an endoluminal stoma channel with an outer ring smaller than the inner ring connected with a conical shaped sleeve.

FIG. 6C is a perspective view of an endoluminal stoma channel with an outer ring roughly the same size as the inner ring connected with a sleeve shaped with a central taper.

FIG. 7 is a schematic sectional view showing an endoluminal stoma channel with a compressed inner ring inserted into the stoma for placement of the device.

FIG. 8A is a schematic sectional view showing an endoluminal stoma channel with a compressed inner ring within an introducer lumen for facilitating placement of the device.

FIG. 8B is a schematic sectional view showing an endoluminal stoma channel with an inflated inner ring and with a deflated inner ring depicted in dashed lines.

FIG. 8C is a schematic sectional view showing an endoluminal stoma channel with a compressed inner ring with a tubular introducer having a plunger inserted therein for facilitating placement of the device.

FIG. 9 is a schematic sectional view showing an endoluminal stoma channel with a distorted inner ring engaged with a delivery tool having a gripping element at its end.

FIG. 10 is a schematic sectional view showing an endoluminal stoma channel in a deployed position within a stoma.

FIG. 11 a schematic sectional view showing an endoluminal stoma channel with a plurality of approximately equal sized inner rings.

FIG. 12 is a schematic sectional view showing an endoluminal stoma channel with a plurality of inner rings with decreasing diameters for rings closer to the stoma opening outside of the patient.

FIG. 13 is a perspective view of an endoluminal stoma channel having a tether connected to the inner ring and configured exterior to the sleeve.

FIG. 14 is a perspective view of an endoluminal stoma channel having a tether connected to the inner ring and configured interior to the sleeve and extending through the outer ring.

FIG. 15 is a schematic sectional view depicting the removal of the endoluminal stoma channel of FIG. 13 through pulling on the tether/pull-string.

FIG. 16 is a schematic sectional view depicting the removal of the endoluminal stoma channel of FIG. 14 through pulling on the tether/pull-string.

FIG. 17 is a front view of an abdomen with an endoluminal stoma channel deployed in a stoma and an adjacent collection bag with a connector element suitable to engage the outer ring of the endoluminal stoma channel, with a figure insert depicting an optional place to engage the outer ring to provide a connection element suitable to engage the bag.

FIG. 18A is a perspective view of an endoluminal stoma channel with an inflatable plug positioned adjacent to and past the inner ring.

FIG. 18B is a perspective view of the devices of FIG. 18A with the plug inflated and positioned to block entry into the inner ring from the bowel.

FIG. 19A is a fragmentary, perspective view of an outer ring and portion of sleeve of an endoluminal stoma channel with a snap engaging outer cover positioned adjacent the outer ring, in which the outer cover is shown with optional valve and optional vent.

FIG. 19B is a fragmentary, perspective view of an outer ring and portion of sleeve of an endoluminal stoma channel with a hinged outer cover connected to the outer ring.

FIG. 19C is a perspective view of the outer cover with a drain tube extending through the vent.

FIG. 19D is a perspective view of an outer cover containing a trapdoor with a flap valve on the inner aspect of the trapdoor.

FIG. 19E is a side view of the outer cover of FIG. 19D with a drainage tube extending through the trapdoor.

FIG. 20A is a perspective view of an inner colostomy bag detachably attached to the outer ring.

FIG. 20B is a perspective view of the inner colostomy bag detachably attached to the outer ring and positioned in the inner aspect of the sleeve and an outer cap which can be detachably attached to the outer ring.

FIG. 20C is a perspective view of the device with the outer cap detached from the outer ring and stool filling the inner colostomy bag thereby extruding the inner colostomy to the outer aspect of the device.

FIG. 20D is a perspective view of inner colostomy bag detached from the outer ring for disposal without spillage.

FIG. 21 depicts a side view of a double-barreled ostomy device which may be used for loop colostomies and ileostomies.

FIG. 22A depicts the inner colostomy bag and a malleable colostomy ring at an open end of the inner colostomy bag and a tab or string located on the periphery of the colostomy ring.

FIG. 22B depicts a side view of the stoma device with the colostomy ring deployed beneath the outer ring and in sealing engagement with the outer ring.

FIG. 23 is a photograph of a patient's stoma and abdomen prior to placement of an endoluminal stoma channel.

FIG. 24 is a photograph of the patient's stoma with an endoluminal stoma channel partially inserted.

FIG. 25 is a photograph of the patient's stoma with an endoluminal stoma channel inserted into the stoma and an outer ring folded over the stoma to cover the stoma.

### OVERVIEW OF THE DRAWINGS

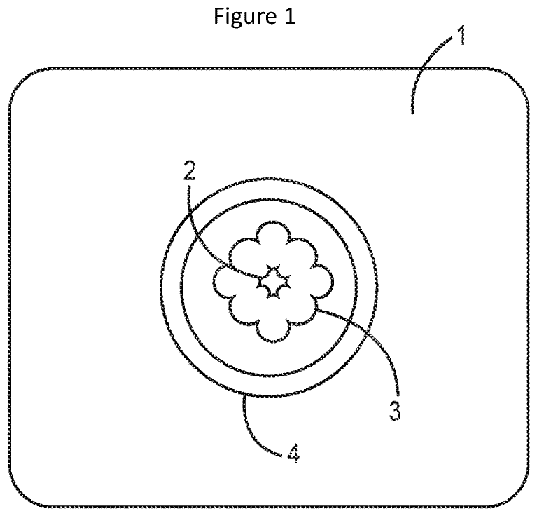

### (FIG. 1)

A prior art colostomy plate 1 with the lumen of the colon 2 which is located in a hole 3 located in the middle of the colostomy plates. Around the periphery of the hole 3, a plastic ring 4 is located where the colostomy bag may be detachably attached to the stoma plate and replaced for emptying and cleaning, etc.

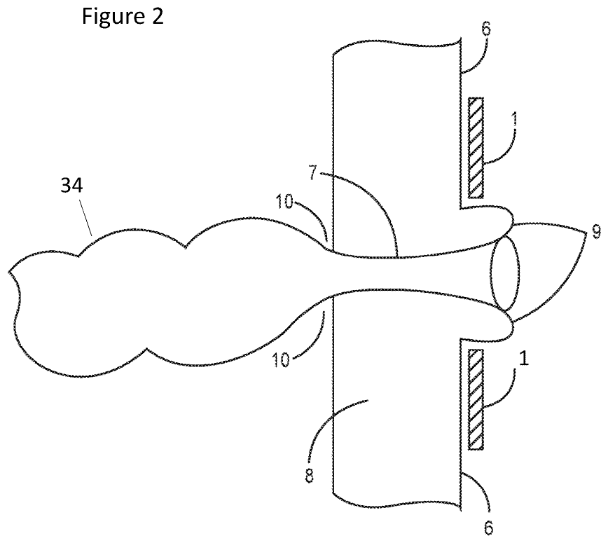

### (FIG. 2)

A transverse view of a colostomy showing the colostomy plates 1 which are adherent to the skin of the abdominal wall 6. The bowel [colon or ileum] is tunneled 7 through the abdominal wall 8 to create a colostomy or ileostomy 9. The point where the colostomy 9 enters the abdominal wall tunnel is referred to as the sub-fascial space 10 and is typically the tightest and most narrowed part of the stoma.

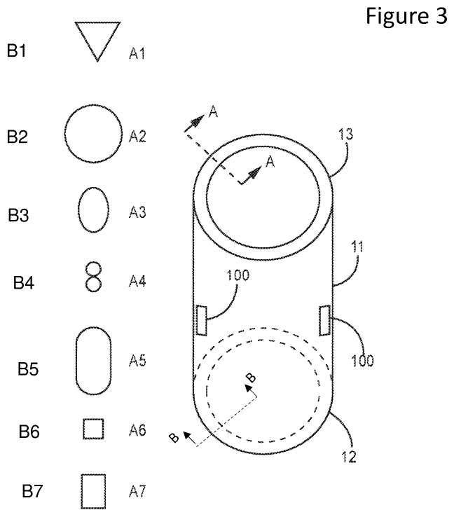

### (FIG. 3)

The device is generally a flexible, elastic generally cylindrical sleeve 11 with two rings attached at each end made of flexible, or malleable or semi-rigid plastic or polymer. An inner ring 12 is located within the lumen of the stoma and lodges in the sub-fascial space 10. The inner ring 12 may be rolled upon itself so shortening the length of the sleeve 11. The inner ring 12 may be prevented from “unrolling” by a “split ring” configuration having a non-circular cross-section area similar to a figure of 8 or other shape illustrated in B1-B7. In a further embodiment both the outer ring 13 and the inner ring 12 may be rolled upon itself so shortening the length of the sleeve membrane 11.

The outer ring 13 may be prevented from “unrolling” by a “split ring” configuration having a non-circular cross-section area similar to a figure of 8 or other shape illustrated in A1-A7. The sleeve 11 may be transparent to visualize the bowel mucosa to ensure it is pink and viable or opaque, non-elastic or elastic.

Referring to FIG. 3, the current device may comprise a generally cylindrical plastic sleeve 11 with an inner ring 12 and outer ring 13 attached to the membrane 11, such as through adhesive bonding, heat bonding, or other suitable procedure. Representative cross-sectional shapes for either inner ring 12 or outer ring 13 are shown in FIG. 3, B1 through B7 and A1 through A7; triangular, circular, oval, figure of 8, elongated curved, square or rectangular. Either ring can be prevented from “unrolling” by a “split ring” configuration having a cross-section area similar to a figure of 8, similar to A4 and B4. Alternatively, the cross-sectional profile may be ovoid, triangular, or polyhedral. This facilitates rolling either ring over and over itself, toward the other ring and thereby making the length of the central sleeve 11 shorter.

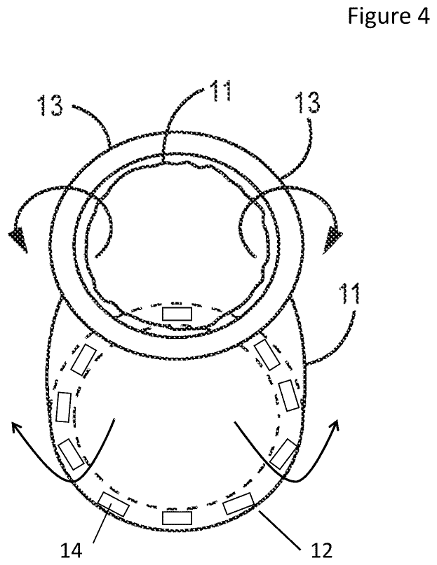

### (FIG. 4)

The cylindrical sleeve 11 is made shorter by rolling the outer ring 13 and/or inner ring 12 upon itself. The diameter of the outer ring 13 is sufficient to cover the external ileostomy or colostomy and facilitates the placement of colostomy bags, occlusive plates and irrigation systems. Sleeve 11 may be made of transparent plastic material to ensure the bowel is pink and viable, and not dusky and ischemic.

In a further embodiment, inner ring 12 is made of a softer and more malleable material in order to minimize pressure on the underlying small bowel or large bowel mucosa and to prevent erosions or ischemia on the bowel wall.

In a further embodiment the inner ring 12 contains circumferential protrusions 14 such as ribs, bumps, ridges, or other suitable shape in order to increase contact between the inner ring 12 and the small or large bowel, to prevent the inner ring 12 from slipping out of the stoma.

Referring to FIG. 4, the outer ring 13 or inner ring 12 are rolled upon itself either inwardly or outwardly thus rolling up the central sleeve 11 around the circumference of the outer ring 13 or inner 12 ring. The central sleeve 11 is thereby shortened. In further alternative embodiments, sets of devices can be sold with various lengths of the central sleeve 11 so that the size can be selected for use with a particular patent. Similar, devices (Alexis retractor, Applied Medical CA) are used to provide exposure for incisions in the abdomen or during cesarean section, but these devices are designed to retract abdominal incisions, rather than as stoma devices.

The sleeve 11 may be transparent, translucent, or opaque in order to visualize the color of the stoma ensuring that it remains pink and viable and not dusky and ischemic.

In a further embodiment inner ring 12 and/or outer ring 13 are made of a softer and more malleable material in order to minimize pressure on the underlying small bowel or large bowel mucosa and to prevent erosions or ischemia on the bowel wall.

The sleeve 11 may be non-elastic or elastic, although the sleeve 11 is generally flexible and tear resistant. Elastic or “stretchable” membranes facilitate deployment and securing the device. The sleeve 11 may comprise a biocompatible polymer. Various biocompatible polymers are known in the art, such as latex, polyesters, polyamines, vinyl polymers, and the like. The sleeve 11 may be fenestrated or contain perforations or holes to allow mucous to enter the lumen of the bowel. The length of the endoluminal device between planes of the rings with the inner and outer rings gently pulled away from each other may be about 0.5-20 cm, in some embodiments about 3-18 cm and in further embodiments about 5-16 cm total length. The cross-sectional diameter of the individual inner and outer rings may be about 0.05-2 cm, in other embodiments about 0.1-1 cm and in further embodiments about 0.25-0.5 cm. A person of ordinary skill in the art will recognize that additional ranges of dimensions within the explicit ranges above are contemplated and are within the present disclosure.

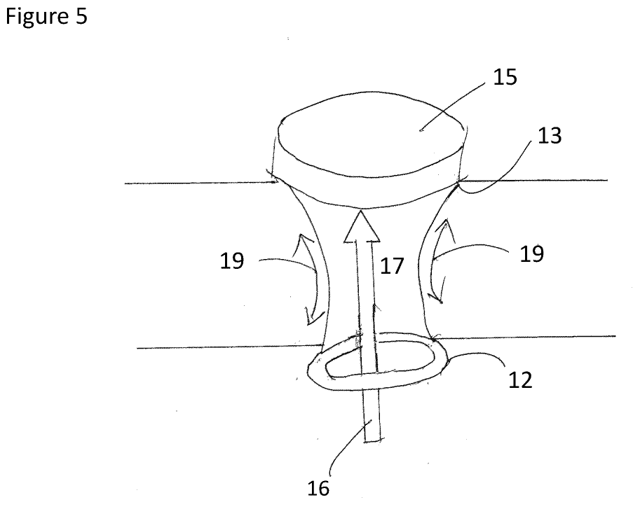

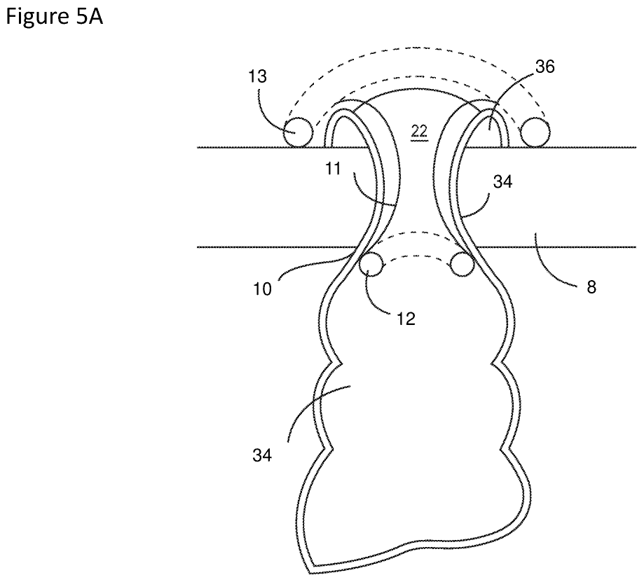

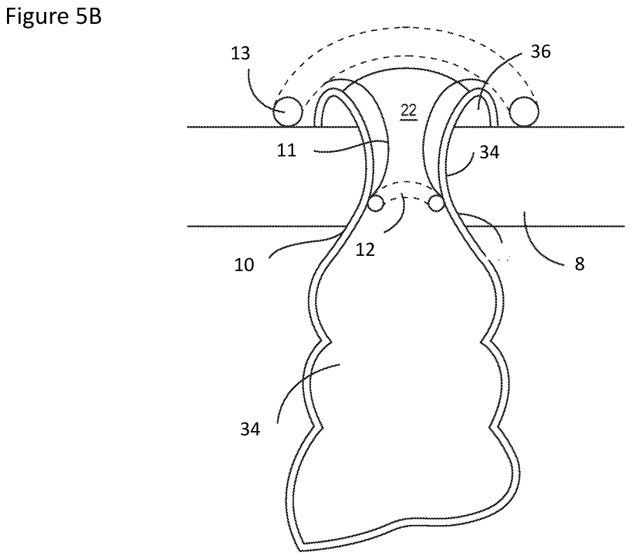

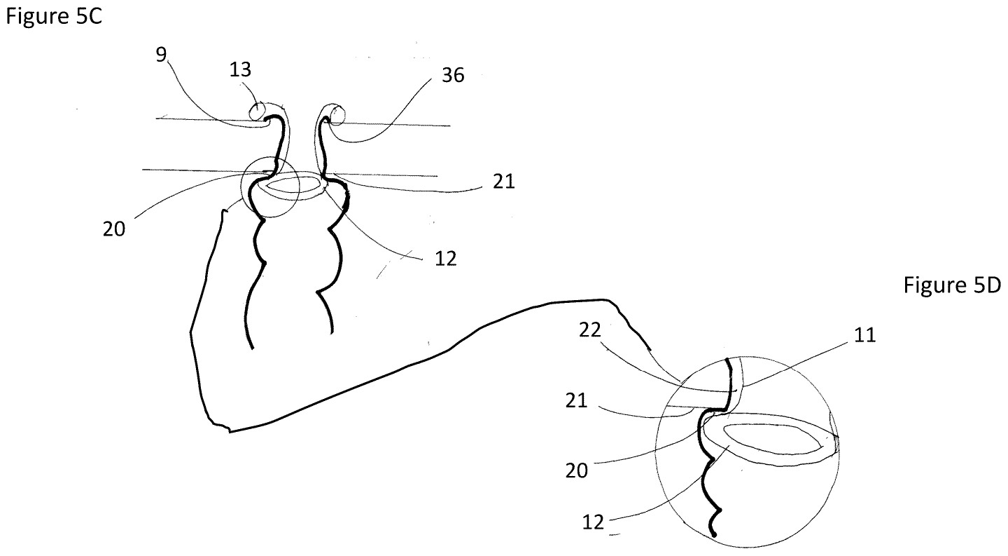

### (FIG. 5)

The inner ring 12 and the outer ring 13 interconnected by the sleeve 11 functions like a shock absorber. Any sudden increase in intra-abdominal pressure 16 is transmitted through the center 17 of the device to the outer cover 15 placing transient tension 19 on the elastic sleeve 11.

Referring to FIG. 5, the dynamic nature of the sleeve 11 and the inner ring 12 and outer ring 13 dampens any excessive intra-abdominal force 16, such as during sudden coughing or sneezing. Any sudden increase in intra-abdominal force 16 puts immediate pressure on the outer cover 15 of the device. Plastic sleeve is operable to absorb transient tension 19 that occurs during any sudden increase in pressure thereby acting as a shock absorber between the inner and outer ring to lessen the chances of extrusion of the inner ring.

### (FIG. 5A)

Transverse section of a stoma with the device located in the lumen. The bowel 34 is tunneled through the abdominal wall 8 to make an external stoma 36 which protrudes above the skin level. The inner ring 12 of the device is located within the lumen of the bowel 34 and gently pulled distally until it lodges in the sub-fascial space 10. The outer ring 13 is rolled over upon itself so shortening the length of the sleeve or membrane 11 which keeps the inner ring 12 secured in the sub-fascial space 10. The outer ring 13 also covers the external stoma 36 to protect it, and to facilitate attachment of the bags or other structures described herein to the outer ring 13.

### (FIG. 5B)

In obese individuals with thick abdominal walls 8, or when the sub-fascial space 10 is abnormally wide, as in para-stomal hernias, the inner ring 12 may deploy at a narrowing of the bowel 34 closer to the surface in the lumen than the sub-fascial space 10, or anywhere within the boundaries of the abdominal wall 8. The endo-luminal device therefore creates a tunnel 22 through the abdominal wall 8 and provides a stable platform to place bags or other structures.

### (FIGS. 5C and 5D)

Any increase in intra-abdominal pressure 16, such as by coughing or sneezing, increases the efficiency of the seal between the inner ring 12, the inner circumference of the stoma 20, and the underside of the abdominal wall 21 decreasing the chance of leakage from around the inner ring 12 and spillage of stool from the stoma.

Referring to FIGS. 5C and 5D, any increased tension on the membrane 11 will also increase the pressure on the inner ring 12 which will force it more securely onto the inner circumference of the stoma 20 and the underside 21 of the abdominal wall. This increased pressure will therefore increase the sealing pressure between the inner ring 12, the inner circumference of the stoma 20 and the underside of the abdominal wall 21 thereby preventing leaks of fluids around the inner ring 12 along the outer surface 23 of the membrane 11, and thus preventing leaks from the stoma.

A further aspect of the current invention involves the mechanical stability between the inner ring 12 and outer ring 13 provided by gentle traction on the circular membrane 11 which gently squeezes the inner aspect of the stoma 20 and the underside of the abdominal wall 21 keeping the stoma device securely in place. The inner ring 12 and outer 13 ring of the device, together with tension in the membrane 11 sandwich the abdominal wall, creating a stable mechanical configuration. This creates a seal between the inner ring 12 and the circumference of the stoma 20 immediately beneath the abdominal wall. This contrasts with the adynamic and rigid configuration of the cylindrical stoma device described in U.S. Pat. No. 4,137,918 to Bogert, which would be easily “shot” out of the stoma during a sudden increase in intra-abdominal pressure, such as during a cough or sneeze.

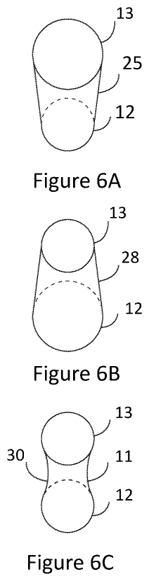

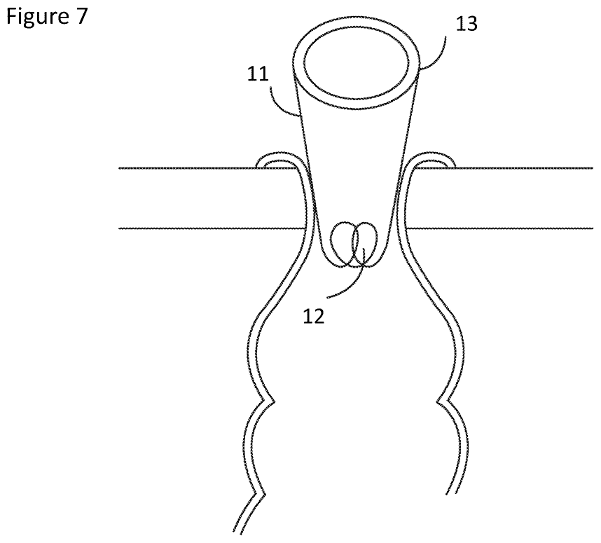

### (FIG. 6A)

The device may be generally cylindrical in form or alternatively conical. The inner ring 12 may be smaller than the outer ring 13, so creating an inverted conical shape 25. The inner ring 12 is located within the lumen of the bowel and the outer ring 13 is located on the abdominal wall of the patient.

### (FIG. 6B)

In an alternative scenario, the inner ring 12 may be larger than the outer ring 13. This then forms a cone shape 28.

### (FIG. 6C)

In an alternative embodiment, the membrane 11 may be tapering at its center 30 with a thinner central waist compared to the inner ring 12 and the outer ring 13.

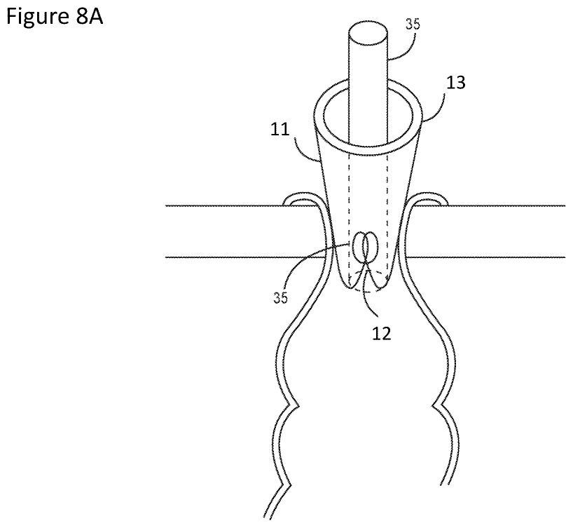

### (FIG. 7)

The inner ring 12 is delivered into the lumen of the bowel by folding the inner ring 12 into an elliptical form, or twisting into a figure of 8 and again upon itself to form a circular form. The inner ring 12 is then inserted into the lumen of the bowel. A portion of the membrane 11 and the outer ring 13 remains above the abdominal wall.

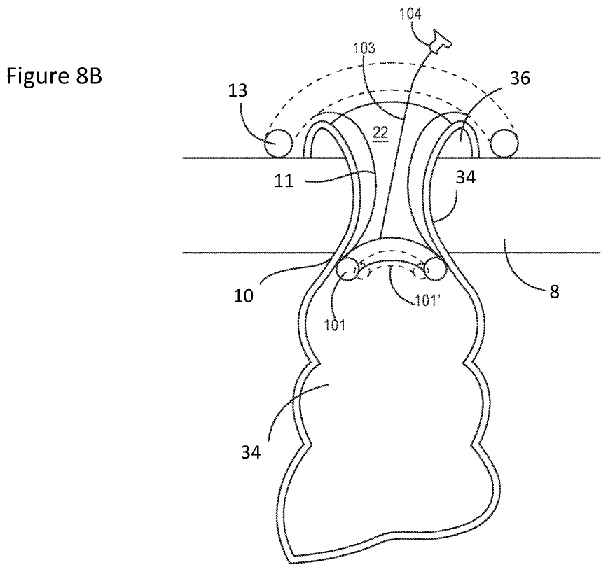

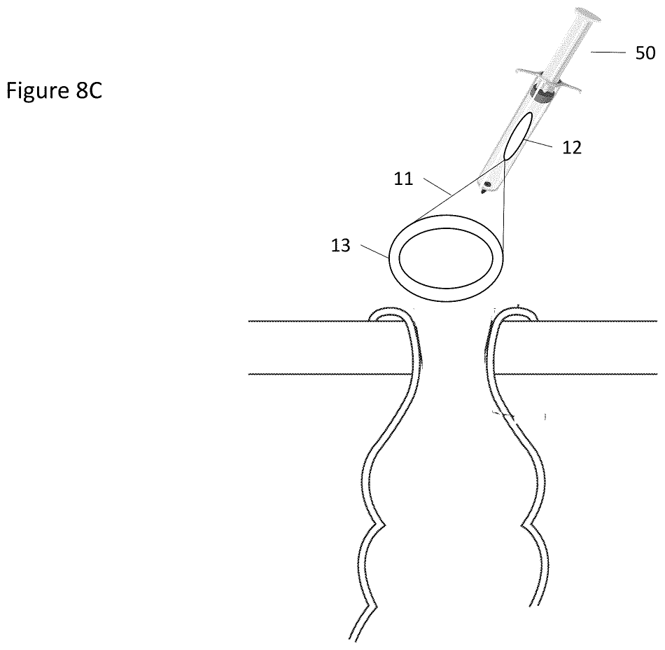

### (FIG. 8A)

A delivery device or introducer 35 may comprise a generally tubular device, which may be non-flexible or flexible. Alternatively, a flexible endoscope or the like may be used to insert the inner ring 12. The inner ring 12 is folded into a figure of 8 and compressed, and is inserted into the distal opening of the introducer 35 or around the end of the introducer. 35. The outer ring 13 and membrane 11 are located around the outside of the circumference of the delivery device or endoscope 35. Furthermore, forceps or the like can grip the inner ring 12 in a reduced profile for insertion into the stoma. The delivery device or endoscope 35 is then inserted into the colostomy and proximal to the sub-fascial space. The inner ring 12 is then gently pushed from the lumen of the delivery device or endoscope 35 for instance using a pusher, or rod, the ring 12 unfolds to form a circular form, and the delivery device 35 is removed.

### (FIG. 8B)

The inner ring can be provided as an inflatable ring 101 that can be inserted in an uninflated state where the ring is manipulatable. As in FIG. 8A, the outer ring and membrane are located around the outside of the circumference of the delivery device, and the delivery device can be used for inserting the inner ring into the colostomy and proximal to the sub-fascial space. A fluid conduit 102 extends from inflatable inner ring 101 to a position that will be located external to the patient. Fluid conduit 102 can be connected to a small balloon (not shown) and terminates at a valve 104, such as a Tuohy-Borst valved adapter, that provides for the attachment of a syringe or the like to inflate or deflate the ring through changing the pressure in the enclosed system extending from inner ring 101 to the valve 104. The small balloon can provide an indication of the pressure within the enclosed system and thereby the state of inflation of inflatable ring 101. Outer ring 13 can be made inflatable also if desired using a similar valved structure.

### (FIG. 8C)

The delivery device may be a tubular introducer 50. The inner ring 12 is folded and compressed and inserted into the distal opening of the tubular introducer 50. The outer ring 13 and membrane 11 are located outside of the distal opening of the tubular introducer 50. The tubular introducer 50 is then inserted into the colostomy and proximal to the sub-fascial space. The inner ring 12 is then gently pushed from the tubular introducer 50 using a moveable pusher device, such as a plunger, releasably held at the proximal opening of the tubular introducer 50, the inner ring 12 unfolds to form a circular form, and the tubular introducer 50 is removed.

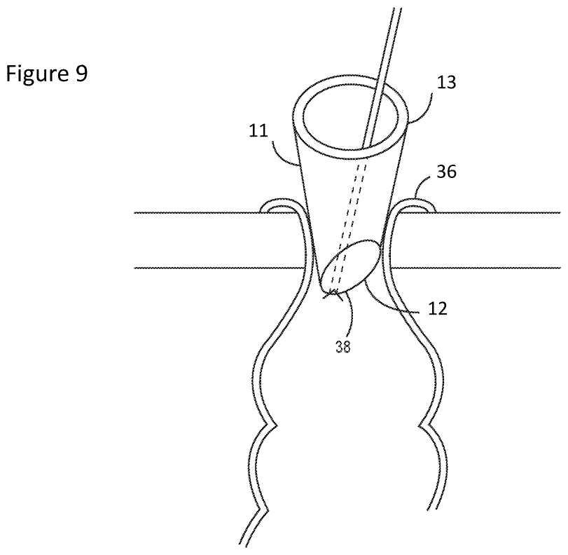

### (FIG. 9)

The inner ring 12 of the device may be introduced into the bowel by applying traction on the inner ring so deforming it into an oval shape. This may be performed by a simple rod shaped device, with a “V” shaped tip 38, passed into the outer ring 13 and sleeve 11 or by applying traction to the pull-string (not shown) of the inner ring 12, for instance by using biopsy forceps of a conventional flexible endoscope inserted into the outer ring 13 and sleeve 11. The inner ring 12 or rings are placed under traction using the V tipped device 38 or by traction on the pull-string which deforms the inner ring 12 or rings into an elliptical shape, which facilitates their placement into the stoma without trauma or pressure, and then released. The biopsy forceps of a conventional endoscope passed through the outer ring 13 and sleeve 11 may be used to grasp the “pull-string” of the inner ring and pull the inner ring into the sub-fascial-space

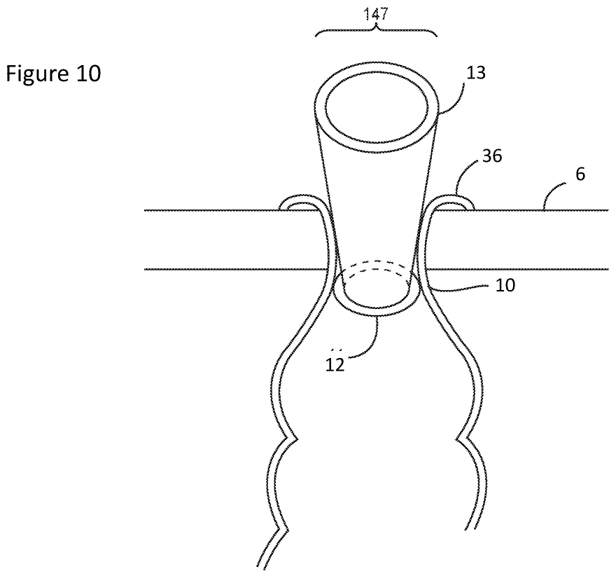

### (FIG. 10)

The inner ring 12 is deployed into the lumen of the bowel and it resumes its previous circular form. It is then gently pulled up into the sub-fascial space 10 where it lodges. Having removed the introducer, the outer ring 13 is then rolled upon itself until it reaches the skin of the anterior abdominal wall 6 and covers or protects the external stoma 36.

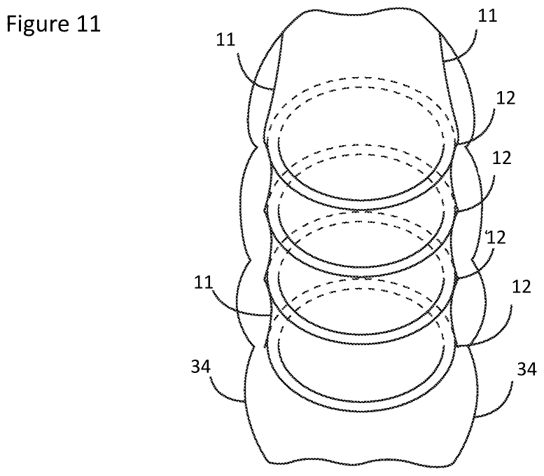

### (FIG. 11)

A plurality of inner rings 12, of equal diameter, are configured in parallel along the sleeve 11, in order to increase traction within the lumen of the bowel 34, to diminish the chances of perforation of the bowel wall.

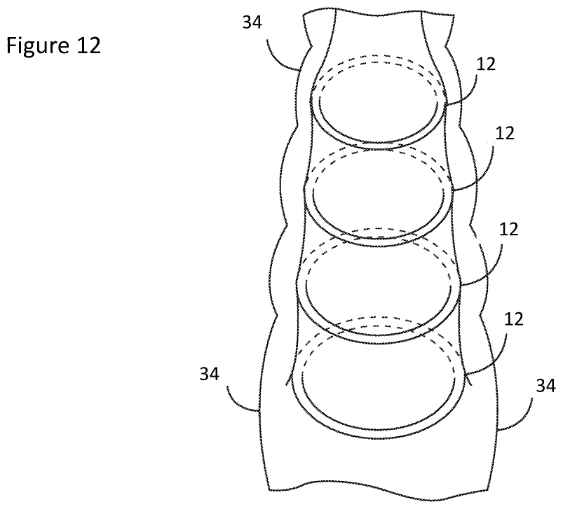

### (FIG. 12)

In a further configuration, the diameter of the inner rings 12 may decrease progressively toward the sub-fascial space. In this configuration peristalsis of the bowel 34 tends to compress the rings together, forming a pyramidal or conical form, so maintaining the inner rings 12 with more stability within the lumen.

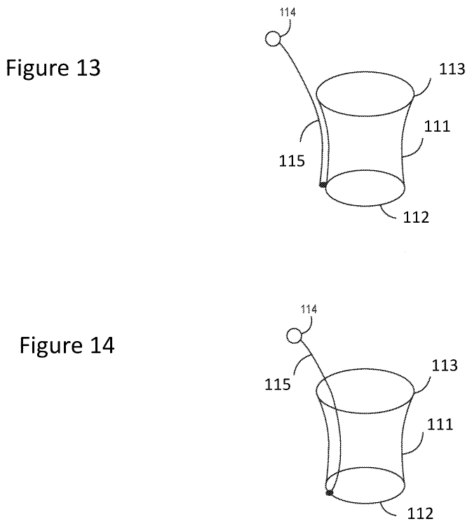

### (FIG. 13)

An embodiment of an endoluminal stoma channel comprises an outer ring 113, inner ring 112, and membrane 111 connecting outer ring 113 and inner ring 112. Tether 115 is connected to inner ring 112 in a configuration to run along the outside of membrane 111. Tether 115 has a knob 114 at its end.

### (FIG. 14)

Alternative embodiment of an endoluminal stoma channel comprises a tether 115 attached to inner ring 112 in a configuration to extend within membrane 111 and to pass through outer ring 113. This embodiment also has a knob 114 at the end of tether 115.

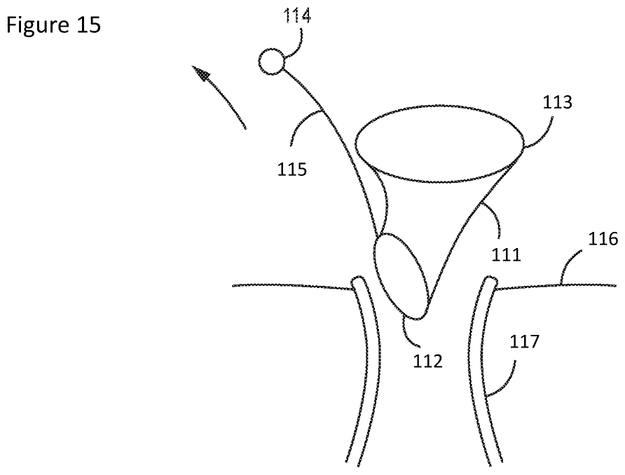

### (FIG. 15)

The removal of the endoluminal stoma channel of FIG. 13 can be effectuated as shown in FIG. 15. Pulling on tether or pull string 115 distorts inner ring 112 such that the device can be removed out from the patient's stoma 117.

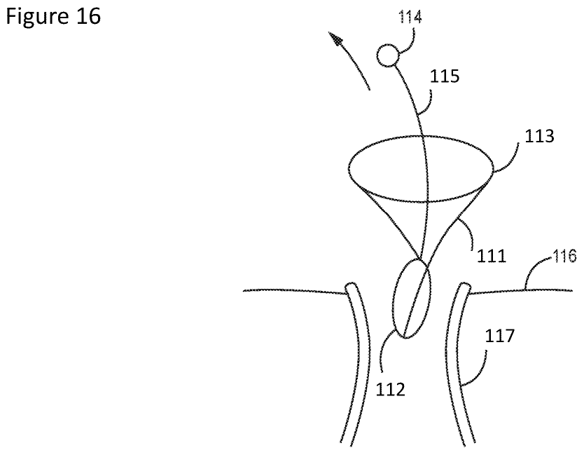

### (FIG. 16)

The removal of the endoluminal stoma channel of FIG. 14 can be effectuated as shown in FIG. 16. Pulling on tether or pull string 115 distorts inner ring 112 such that the device can be removed out from the patient's stoma 117.

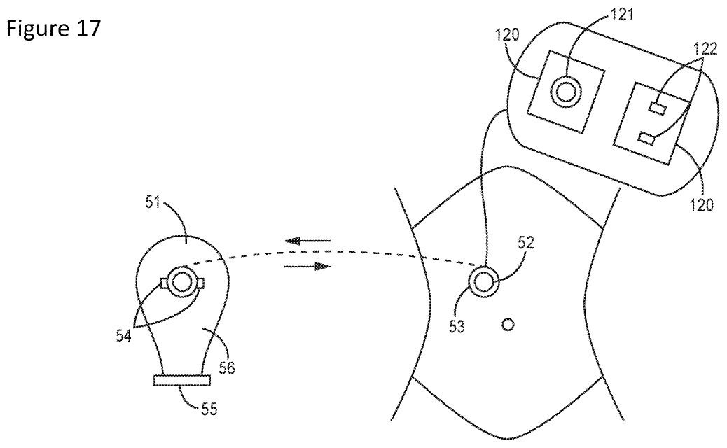

### (FIG. 17)

An embodiment of an attachable bag 51 is shown separate from the colostomy attachment device, endoluminal stoma channel 52. Bag 51 is designed for attachment to the outer ring 53. Bag 51 has a fastener 54 that can comprise a clamp or seal element with a releasable lever to attach and remove the bag or a snap-type design or the like similar to conventional colostomy bags. Bag 51 can have a closable drain 55 with a hook and loop closure or a clamp closure or the like, although in other embodiments the bag is sealed on its end for disposal and replacement of the bag rather than drainage.

### (FIG. 17 Inserts)

An embodiment adapted from the device of FIG. 17 is shown in the inserts of FIG. 17. The endoluminal stoma channel is shown with the outer ring replaced or supplemented with a wafer-type structure 120. The inner ring and sleeve can correspond with any of the embodiments described herein. Wafer-type structure 120 can be permanently attached or releasably attached to an outer ring using similar attachment as described in the context of FIG. 17. Wafer-type structure 120 further comprises a fastener 121 for the attachment of a bag. Fastener 121 can be any reasonable structure such as a snap structure or the like.

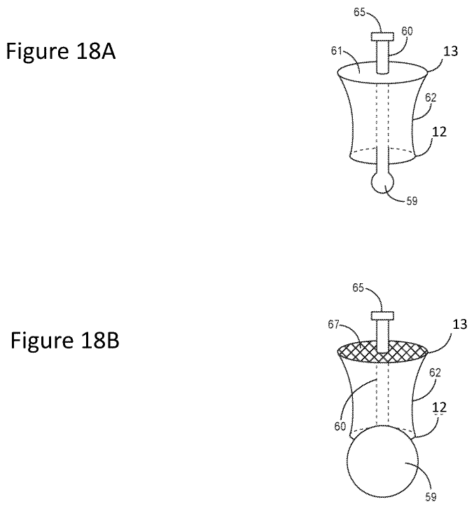

### (FIG. 18A)

In a further embodiment, an inflatable balloon is 59 provided, attached to a fluid conduit 60, which is inflated in a similar manner as the balloon on a Foley catheter. The deflated balloon is inserted through the lumen 61 of the deployed device 62 and positioned proximal to the inner ring 12 of the deployed device. The balloon 59 is then inflated with fluid via the fluid conduit 60. The fluid conduit has a valve 65, such as a luer lock valve, which maintains pressure within the balloon and prevents leakage from the balloon 59.

### (FIG. 18B)

The inflated balloon 59 is pulled gently toward the inner ring 12 of the device to form a seal between the inflated balloon and the inner ring of the device, similar to a “Ball valve” device. The inflated balloon 59 prevents stool, liquid and gas from exiting through the device and creates a stable mechanical seal between the balloon 59 and the inner ring 12. The inflatable fluid conduit 60 may be secured at the outer ring 13 or the plate attached to the outer ring by a slot or key-hole type configuration 67 which secures the balloon 59 at the inner ring 12 and maintains the seal between the balloon 59 and the inner ring 12.

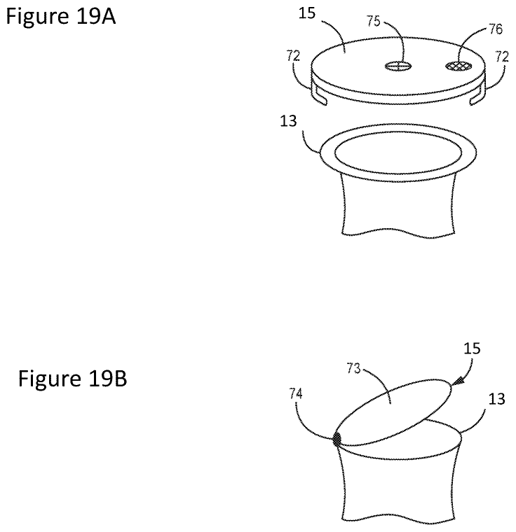

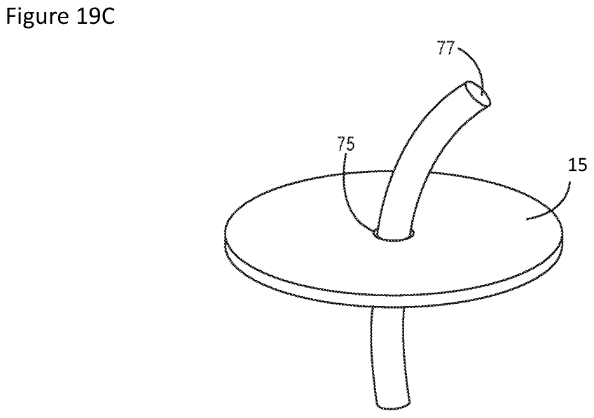

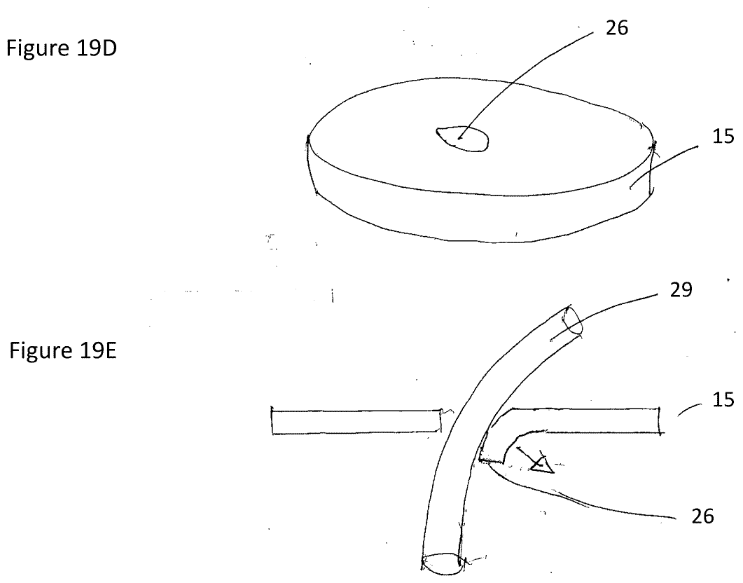

### (FIG. 19A)

The outer cover 15 with clasps 72 is shown adjacent an outer ring 13 configured for connection onto outer cover 15. Optional valve 75 and optional vent 76 are depicted on the surface of cap 70.

### (FIG. 19B)

An alternative embodiment of the outer cover 15 having a hinge 74 connecting outer cover 15 to outer ring 13.

### (FIG. 19C)

The outer cover 15 includes a drain tube 77 extending through valve 75.

### (FIG. 19D)

The outer cover 15 includes a trapdoor 26 with a flap valve on the inner aspect of the trapdoor 26 to prevent leakage of stool or effluent.

### (FIG. 19E)

When drainage tube 77 is inserted through the trapdoor 26, the trapdoor 26 is displaced allowing for the drainage of the contents within the sleeve.

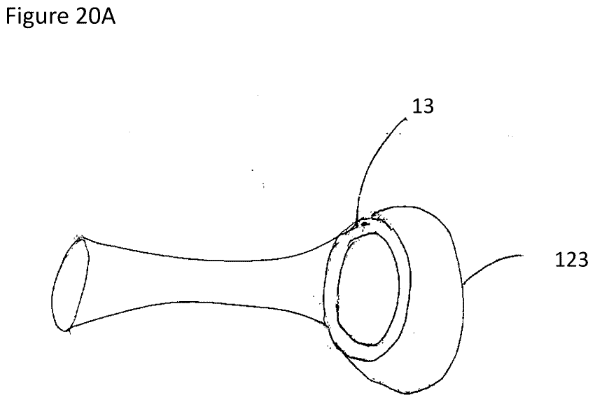

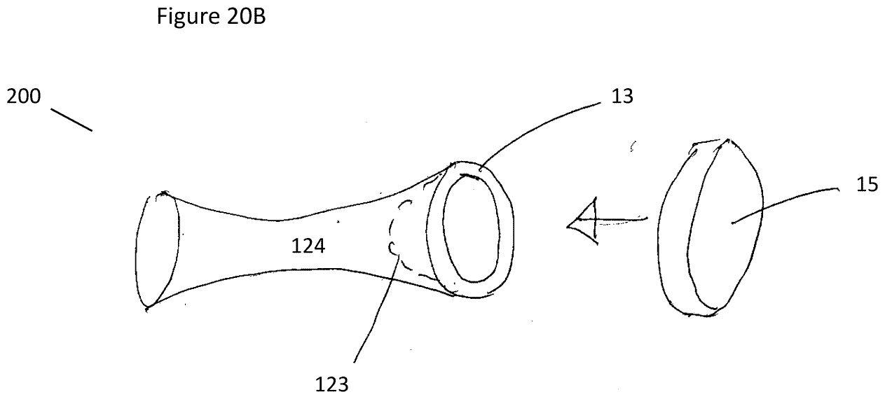

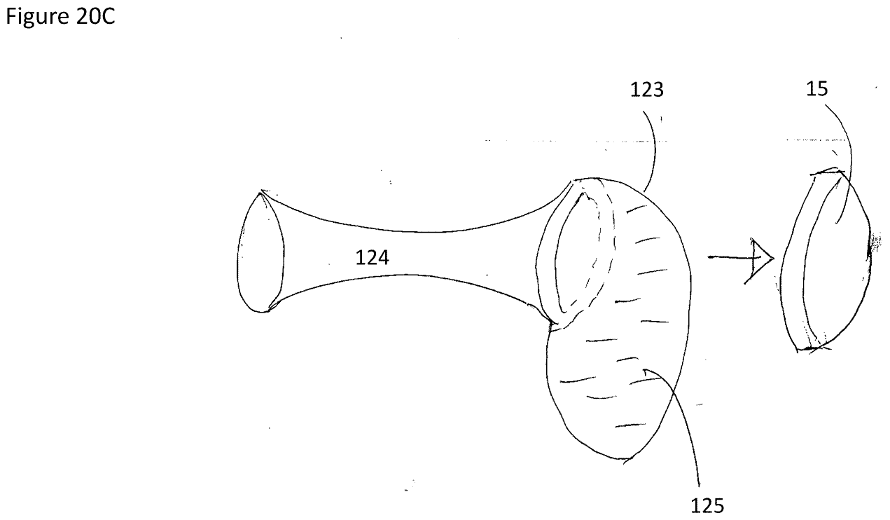

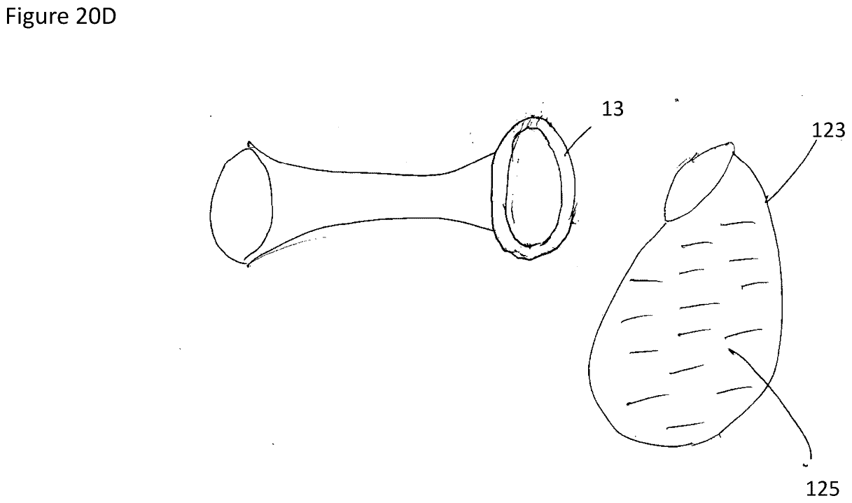

### (FIG. 20A)

An inner colostomy bag or condom 123 releasably attaches to the outer ring 13 to act as an “internal” colostomy bag.

Referring to FIG. 20A, the current embodiment also includes the provision of an inner colostomy bag 123 made of thin walled, high compliance, pliable plastic, similar to a large condom or balloon. The inner colostomy bag 123 may be made of latex, or suitable non-latex polymer, may be biodegradable and may be transparent, translucent or opaque. The inner colostomy bag 123 is releasably attached to the outer ring 13 using a snap-on mechanism, (similar to a Zip-Lock bag or conventional colostomy attachment) or a slip-on mechanism.

### (FIG. 20B)

The inner colostomy bag 123 is releasably attached to or over, or within, the circumference of the outer ring 13 and gently inserted into the inner aspect 124 of the sleeve of the device.

Referring to FIG. 20B, once attached to the outer ring 13, the inner colostomy bag 123 may be gently delivered into the inner aspect 124 of the sleeve of the device. The outer cover 15 is detachably attached to the outer ring 13, securing the inner bag 123 in the inner aspect 124 of the sleeve to seal off the device and prevent drainage of stool.

### (FIG. 20C)

When the outer cap 15 is removed from outer ring 13, stool 125 slowly fills inner colostomy bag 123. The inner colostomy bag 123 is extruded to the outer aspect of the stoma device as the colostomy bag 123 fills, similar to a conventional outer colostomy device.

### (FIG. 20D)

When the inner colostomy bag 123 is full (or at least partially full), it can be detached from the outer ring 13 and disposed of cleanly and without spillage. The inner colostomy bag 123 may be cleaned (or a new bag 12) and is then detachably attached to the outer ring 13 and the outer cover 15 is reattached to the outer ring 13. The inner colostomy bag 123 prevents the free spillage of stool 125 from the stoma device when the outer cap 15 is removed and permits clean and controlled delivery of stool 125 into the inner colostomy bag 123 which can then be disposed of cleanly.

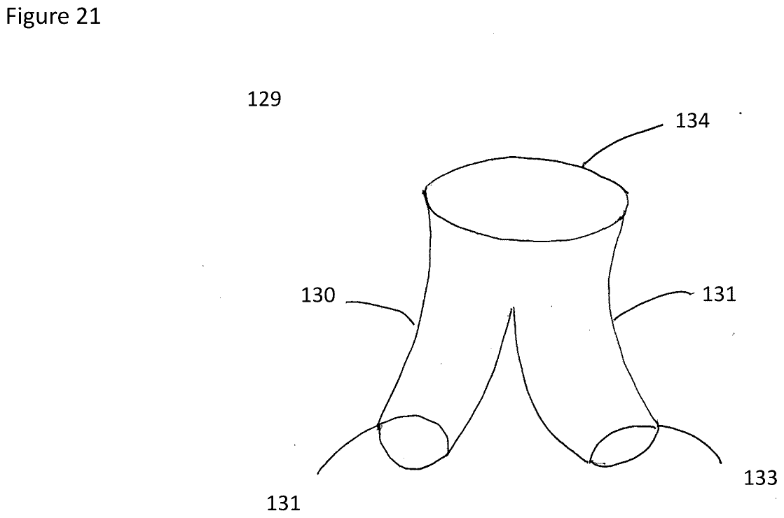

### (FIG. 21)

A double-barreled ostomy device 129 is provided for loop colostomies and ileostomies. The device has two separate limbs 130, 131 one for each loop of the stoma. The inner rings 132, 133 of the two lumen device are inserted in the afferent and efferent limb of the stoma. The two lumens communicate with a common outer ring 134 which can be folded to the abdominal wall in the same manner as the single limb stoma device.

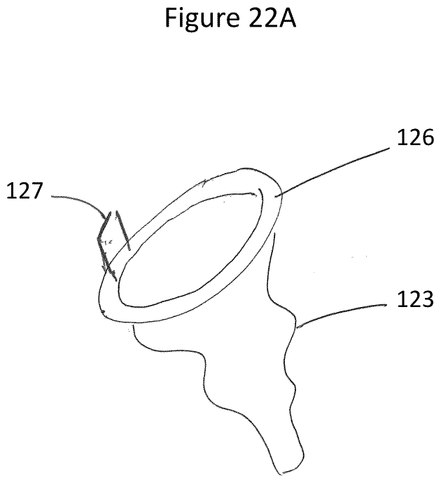

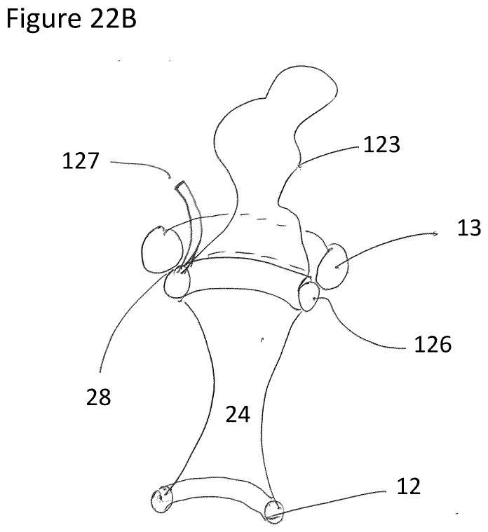

### (FIGS. 22A and 22B)

Referring to FIG. 22A, the inner colostomy bag 123 incorporates a malleable colostomy ring 126, at the bottom end of the inner colostomy bag 123. The malleable ring 126 has similar dimensions and malleable properties as the inner ring 12. The malleable ring 126 can be gently compressed and inserted inside the inner aspect of sleeve below the outer ring 13 to create a seal.

Referring to FIG. 22B, after releasing the colostomy ring 126 from compression, it returns to or close to its prior configuration and deploys beneath the outer ring 13 of the stoma device where it sealingly engages the outer ring 13. (Alternatively, the colostomy ring 126 may be deployed above the outer ring 13 to sealingly engage the outer ring 13). The inner colostomy bag 123 is inserted inside the inner aspect of the stoma device 24, and the outer cap 15 is releasably attached to the outer ring 13.

In a further embodiment, a tab or string 127 may be located on the periphery of the colostomy ring 126 to assist in removing or venting the inner colostomy bag 123. The tab 127 may also be used to release gas from the lumen of the bowel in the event of gas build-up. Applying traction on the tab 127 will deform the colostomy ring 126 breaking the seal between the colostomy ring 126 and the outer ring 13 and allowing for the release of gas or flatus. The colostomy ring can then be returned below the outer ring to sealingly engage the outer ring. Ideally, the tab 127 may be located at a 12 o'clock position on the colostomy ring 126 to preferentially allow for the escape of gas or flatus located on top of the stool surface rather than the escape of stool.

In still a further embodiment, the tab 127 may further be releasably attached to the periphery of the outer cover (not shown) such that partial removal of the outer cover will result in traction on the tab to compress the colostomy ring 126 and break the seal between the colostomy ring 126 and the outer ring 13 resulting in the release of gas. In yet a further embodiment the tab 127 may further protrude through an opening on the outer cover allowing the user to apply gentle traction on the tab 127 to compress the colostomy ring 126 and break the seal with the outer ring 13 resulting in the release of gas without having to remove the outer cover itself. Applying pressure to the tab 127 will return the colostomy ring 126 to the inner aspect 128 of the outer ring 13 to re-seal the inner colostomy ring 126 with the outer ring 13. These configurations would preferably be located at the 12 o'clock position on the colostomy ring 126 and outer cover to preferentially allow for the escape of gas, and not stool.

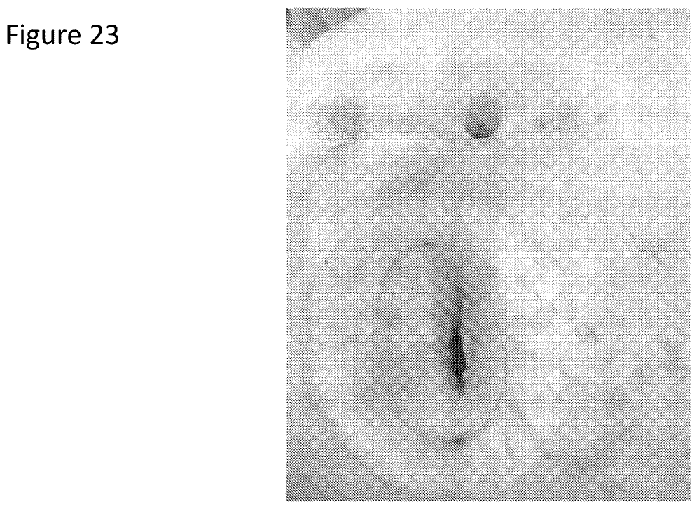

### (FIG. 23)

A 57 year old morbidly obese male undergoes a Hartmanns resection for perforated diverticulitis. Because of his obesity, and a 5 in deep abdominal wall, the end of the colostomy underwent ischemic necrosis, leaving a pencil thin “blow-hole+ fistula.

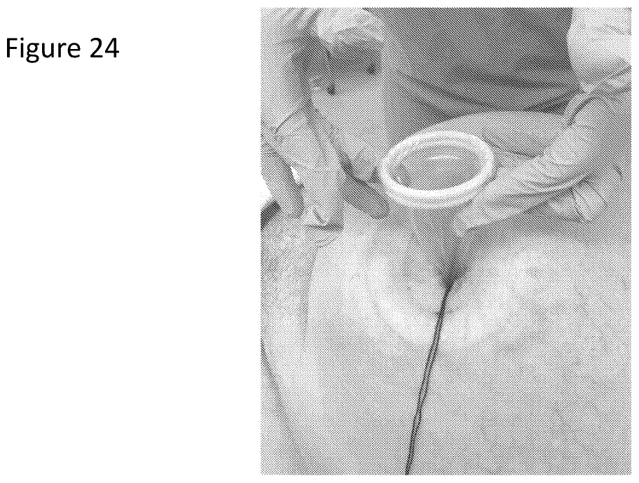

### (FIG. 24)

The device of the present disclosure was inserted into the “blow-hole” colostomy forming a stable platform for placement of a colostomy bag.

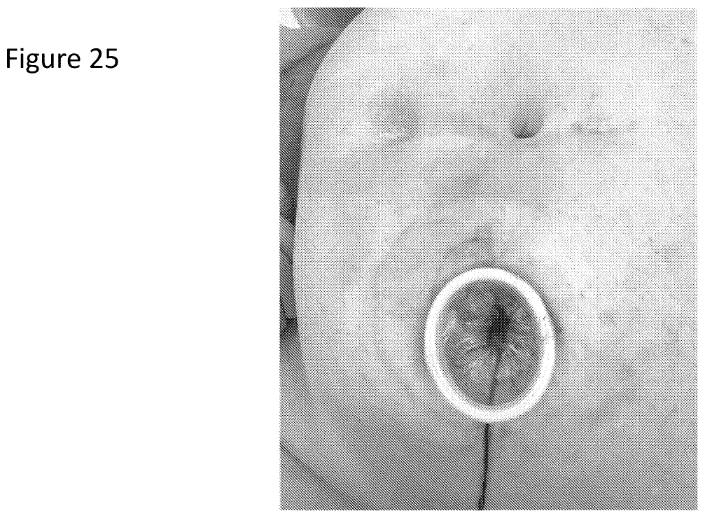

### (FIG. 25)

The device of the present disclosure was stable and remained in place and even applying significant traction to the device. The patient underwent a successful bowel prep to clean out the colon and had surgery to reverse the colostomy.

### DETAILED DESCRIPTION OF THE INVENTION

The devices described herein provide for secure interface with a stoma formed with a colon or ilium (small bowel) by placement of a sleeve extending from the stoma and secured with a ring anchored within the patient. The sleeve can be conveniently placed within the patient to provide a way to control output from the stoma without gluing something to the patient's skin. The ostomy device can be referred to as an endoluminal stoma channel, and these terms are used interchangeably herein for convenience. Various covers are described to secure onto an outer ring to provide desired control and controlled access. The endoluminal stoma channel and a cover as well as other possible components can be referred to as a stoma discharge control system, although the components can be referred to without specific reference to the system. The rings generally can have a circular shape in the plane of the ring, but other shapes can be effective too, especially for the outer ring. For example, the inner ring can have a minor oval shape. While an outer ring with a circular shape can fit snugly over the stoma, other shapes such as a square can also fit over the stoma and may provide different attachment modalities for covers and the like.

In contrast with the stoma anchored devices described herein, referring to FIG. 1 a typical colostomy device consists of an adhesive plate 1 with central hole 2 and through which a colostomy or ileostomy protrudes 3. A clip or seal attachment 4 around the circumference of the hole allows detachment of colostomy bags and the stool empties into the bag. The bag is emptied and cleaned from time to time or replaced.

Referring to FIG. 2, the depiction illustrates a cross-section of a conventional colostomy with a prior art colostomy device of FIG. 1 secured to the patient. The colostomy plate 1 adheres to the skin of the abdominal wall 6. The colostomy bag is attached to the plate using a plastic snap-on or clip-on mechanism similar to a Zip-Lock bag, as known in the art. The bowel itself 34 is tunneled through the anterior abdominal wall 8 and forms an external colostomy or ileostomy depending on whether the colon [colostomy] or ileum [ileostomy] 9 is used. The narrowest part of the stoma is at the sub-fascial space 10, where the bowel enters the abdominal wall 6. Stomas are used after resection of the bowel or when re-anastomosis of the bowel is not feasible, for instance after Abdomino-perineal resection for the rectum for cancer.

Referring to FIG. 3, the current device comprises a generally cylindrical plastic membrane 11 with an inner ring 12 and outer ring 13 attached to the membrane 11, such as through adhesive bonding, heat bonding, or other suitable procedure. The plastic membrane 11 may be fenestrated or contain perforations 100 to allow mucous secretions into the lumen of the bowel. The rings 12, 13 can be made of either flexible, soft or semi-rigid polymer or plastic, spring metal (e.g., Nitinol) or combinations thereof, and the inner ring and the outer ring can be made from the same or different material(s). In one embodiment, the outer ring comprises a first polymer having a first shore durometer value and the inner ring comprises a second polymer having a second shore durometer value where the first shore durometer value generally is greater than the second shore durometer value. The inner ring 12 and/or the outer ring 13 can be inflatable to facilitate placement and securing of the ring, and an appropriate tube with a suitable valve can be located to be accessible outside of the patient to allow for inflating and deflating the ring. Catheter balloons, balloon lumen and appropriate valves are known in the art, such as in association with Foley catheters. In some embodiments, the inner and/or outer rings 12, 13 have a non-circular cross section and can be rolled upon itself to shorten the sleeve membrane. Representative cross-sectional shapes for either inner ring 12 or outer ring 13 are shown in FIG. 3 cross-sections, B1 through B7 and A1 through A7; triangular, circular, oval, figure of 8, elongated curved, square or rectangular. The rings can be prevented from “unrolling” by a “split ring” configuration having a cross-section area similar to a figure of 8, similar to cross-section B4 and A4. Alternatively, the cross-sectional profile may be ovoid, triangular, or polyhedral. This facilitates rolling the outer ring 13 over and over itself, toward the inner ring 12 and thereby making the length of the central membrane 11 shorter. The membrane 11 may be transparent, translucent, or opaque. The membrane 11 may be rigid, flexible, or fully or partially collapsible; expandable, non-expandable, or partially expandable; soft or hard; or any appropriate combination of the foregoing. Moreover, sleeve 11 may be relatively thin walled, e.g., to permit as large of an inner diameter and/or cross-sectional area as possible for the movement of waste, such as fecal matter, urine or mucous through the sleeve 11. The sleeve 11 may be non-elastic or elastic, although is generally flexible and tear resistant. Elastic or “stretchable” membranes facilitate deployment and securing the device. The membrane 11 may comprise a biocompatible polymer. Various biocompatible polymers are known in the art, such as latex, polyesters, polyamines, vinyl polymers, and the like. The membrane 11 may be fenestrated or contain perforations or holes to allow mucous to enter the lumen of the bowel. The length of the endoluminal stoma channel between planes of the rings with the inner and outer rings gently pulled away from each other may be at least about 0.5 cm or at least about 3 cm or at least about 5 cm or at least about 10 cm or at least about 15 cm. In some embodiments, the length of the endoluminal stoma channel between planes of the rings with the inner and outer rings gently pulled away from each other may be less than about 25 cm or less than about 20 cm or less than about 16 cm or less than about 12 cm. In another embodiment, the length of the endo-luminal channel between planes of the rings with the inner and outer rings gently pulled away from each other may be between about 1-20 cm, in some embodiments between about 3-18 cm and in further embodiments between about 5-16 cm total length. The cross-sectional diameter of the individual inner and outer rings may be at least about 0.05 cm or at least about 0.1 cm or at least about 0.5 cm or at least about 1 cm. In another embodiment, the cross-sectional diameter of the individual inner and outer rings may be less than about 2.5 cm or less than about 2 cm or less than about 1.5 cm. In another embodiment the cross-sectional diameter of the individual inner and outer rings may be between about 0.05-2 cm, in other embodiments between about 0.1-1 cm and in further embodiments between about 0.25-0.5 cm. A person of ordinary skill in the art will recognize that additional ranges of dimensions within the explicit ranges above are contemplated and are within the present disclosure.

Referring to FIG. 4, the inner and/or outer rings 12, 13 may be rolled upon itself either inwardly or outwardly thus rolling up the central membrane 11 around the circumferences of the inner and/or outer rings 12, 13. The central membrane 11 is thereby shortened. In further alternative embodiments, sets of devices can be sold with various lengths of the cylindrical membranes 11 so that the size can be selected for use with a particular patient. In additional or alternative embodiments, devices can be sold so that the length of the membrane 11 can be adjusted and clipped at a certain length, with or without rolling the inner and/or outer rings, to achieve a selected membrane length for the patient prior to deployment. In an alternative embodiment, the outer ring 13 may be detachably attachable to the cylindrical membrane 11, so the length of the cylindrical membrane 11 may be cut to length, according to the body habitus of the patient (long length of cylindrical sleeve 11 needed for obese patients; shorter lengths for thinner patients), and then clipped to the ring. Similar, devices (Alexis retractor, Applied Medical CA) are used to provide exposure for incisions in the abdomen or during cesarean section, but these devices are larger and designed for different techniques for delivery and removal.

In one embodiment, inner ring 12 is resilient and deformable with an outer surface that is generally flat around a circumference. In another embodiment, the inner ring 12 is resilient and deformable, and its undeformed configuration has an outer surface comprising contours and/or texture to present contact points of polymer having a shore durometer value of no more than 65 A. Thus, in one particular embodiment, the outer ring can comprise a polymer having a shore durometer value greater than 65 A and in further embodiments greater than about 30D. The contours and/or texture may take any form or shape as desired for the particular operation or intended use. For example, the outer surface of inner ring 12 may have one or more concave, ogee, French curve, arch, or hook regions. In other embodiments, the outer surface of inner ring 12 may be textured for example, it may comprise one or more nubs, bumps, ribs, or protrusions that may take any shape, such as round, square, circular/circle, rectangular/rectangle, triangular/triangle, cylindrical/cylinder, elliptical/ellipse, or (n)polygonal/(n)polygon where n is an integer of at least 5. In some embodiments, the contact points of polymer may have a shore durometer value of no more than about 60 A, or no more than about 55 A, or no more than about 50 A, or no more than about 45 A, or no more than about 40 A, or no more than about 35 A, or no more than about 30 A, or no more than about 25 A, or no more than about 20 A, or no more than about 15 A, or no more than about 10 A In one aspect of this embodiment, the contact points of polymer may have a shore durometer value in a range that includes and/or is between any two of the foregoing. For example, the contact points of polymer may have a shore durometer value Shore of about 10 A to about 65 A. A person of ordinary skill in the art will recognize that additional ranges of Shore duromater hardness within the explicit ranges above are contemplated and are within the present disclosure.

The dynamic relationship between the sleeve 11 interconnecting the inner ring 12 and the outer ring 13 is depicted in FIG. 5. Any sudden increase in intra-abdominal pressure 16, such as from coughing or sneezing, will be transmitted through the center 17 of the device to the outer cover 15 placing transient tension 19 on the elastic sleeve 11. Because of its elasticity, the sleeve 11 can absorb and dampen these sudden spikes in pressure, in effect functioning like a shock absorber, and reducing the chances of the inner ring 12 being extruded.

FIG. 5A illustrates a cross-section of a colostomy or ileostomy of the device positioned in place. The colon 34 or ileostomy is tunneled through the anterior abdominal wall 8 and an external stoma 36 is created by everting the cut edge of the bowel to form a spout. This facilitates placing a colostomy plate and avoids ulceration or digestion of the skin around the stoma. The inner ring 12 of the device has a diameter either equal to, or slightly larger than the diameter of the inner lumen of the small bowel or colon of a human. Generally, this diameter is between about 0.1-10 cm, in some embodiments between about 0.5-7 cm, in further embodiments between about 1-6 cm diameter. The diameter of the bowel may vary widely in certain pathological conditions, and various sizes of device can be available, depending on the individual stoma (for instance, small; medium and large). Upon delivery into the stoma, the inner ring 12 can be pulled gently toward the sub-fascial space 10 by applying gentle traction to the outer ring 13. The sub-fascial space 10 is typically the narrowest part of the stoma, since the bowel is constricted as it enters the tunnel through the abdominal wall 8 and provides a ledge where the inner ring 12 lodges.

The outer ring 13 can be rolled upon itself and the length of the sleeve membrane 11 is thereby shortened. The outer ring 13 may have a diameter equal to or larger than the inner ring 12. The diameter of the outer ring is generally between about 1-12 cm, in some embodiments between about 2-10 cm in diameter, and in further embodiments between about 3-8 cm. A person of ordinary skill in the art will recognize that additional ranges of inner ring diameters and outer ring diameters within the explicit ranges above are contemplated and are within the present disclosure. As the outer ring 13 can be rolled upon itself, it deploys circumferentially around the outer stoma 36 so protecting it from drying or injury. The outer ring 13 then creates a stable platform where a stoma bag or occluding plate may be detachably attached using a clip mechanism or flip-on mechanism. The need for plates, glues and adhesives is therefore avoided, and erosion breakdown or ulceration of the skin which is often seen in current colostomies can be avoided.

For a configuration with a ring deployment not as deep in the stoma, referring to FIG. 5B, the inner ring 12 may be deployed at a site in the lumen closer to the surface than the sub-fascial space 10. This may occur in obese individuals with very deep abdominal walls 8, or in the presence of an abnormally enlarged sub-fascial space 10 as occurs in the presence of a para-stomal hernia. For instance, the inner ring 12 may deploy at a site or narrowing 14 within the boundaries of the abdominal wall 8, between the sub-fascial space 10 and the external stoma 36. In this scenario, the inner ring 12 lodges at the narrowest part of the lumen 14. In this scenario, radial traction from the inner ring 12 maintains the inner ring 12 in place.

Referring now to FIGS. 5C and 5D, as discussed above, any increase in intra-abdominal pressure 16, such as during coughing or sneezing, will increase tension in sleeve 11 allowing the sleeve 11 to provide sufficient tension on the inner ring 12 to anchor the inner ring 12 more securely onto the inner circumference 20 of the stoma and the underside 21 of the abdominal wall. Such tension applied to the inner ring 12 from the sleeve 11 is sufficient to prevent waste material and/or gas from leaking around the inner ring 12 along the sleeve's outer surface 23 which, if not present, would result in spillage from the stoma.

A further aspect involves improved mechanical stability between the inner ring 12, outer ring 13 and sleeve 11. The outer ring 13 is configured to cover the external stoma and to provide an anchoring structure for the sleeve 11. The sleeve 11 is composed of an elastic biocompatible polymer having an intrinsic tension. Because the two rings 12 and 13 are integrated into the sleeve 11, each erect themselves proximally and distally to the sleeve due to the elastic intrinsic tension thereof. Thus, the outer ring 13, sleeve 11 and inner ring 12 in effect, sandwich the abdominal wall thereby creating a stable mechanical configuration.

Referring to a representative embodiment in FIG. 6A, the inner ring 12 may be smaller than the outer ring 13 as in an inverted cone 25 configuration. This allows for the inner ring 12 to be located within the lumen of the small bowel or large bowel and the outer ring 13 may be large enough to cover the external aspect of the colostomy or ileostomy. FIG. 6B illustrates an embodiment with an upright cone where the inner ring 12 is larger than the outer ring 13 and the central membrane 11 is in the form of an upright cone. FIG. 6C is another alternative configuration where the inner membrane 11 may be in the form of a narrow waist 30 where the central portion of the membrane 11 is narrower than the inner ring 31 or the outer ring 32.

Referring to FIG. 7, the inner ring 12 is deployed into the lumen of the bowel by folding the semi-rigid or elastic inner ring into a figure of 8 and then folding it upon itself once again to form a circular configuration. This makes the diameter of the inner ring smaller and easier to introduce into the lumen of the bowel. The inner ring 12 is then inserted into the lumen of the bowel, such as manually or with forceps, and the membrane 11 and the outer ring 13 protrude above the abdominal wall.

Referring to FIGS. 8A and 8C an introducer or endoscope 35 may be used to introduce the folded inner ring 12 into the stoma. The introducer 35 may be solid or tubular (as shown in FIG. 8C), and either non-flexible or flexible, with dimensions generally from about 0.5-8 cm or in further embodiments about 1-2 cm in diameter so it can be inserted into the lumen of the colostomy or the ileostomy. A person of ordinary skill in the art will recognize that additional ranges of introducer diameters within the explicit ranges above are contemplated and are within the present disclosure. The inner ring 12 is compressed into a suitable distorted form and introduced into the lumen by the introducer 35. Alternatively, a conventional upper endoscope or flexible endoscope may be used to insert the device. In one embodiment, the inner ring 12 is twisted into a figure of 8, then into a circular form and placed into or around the distal aspect of the introducer or endoscope 35. The membrane 11 and outer ring 13 are located around the circumference of the introducer or endoscope 35 as it is inserted into the stoma. The introducer or endoscope 35 is inserted into the lumen of the colostomy or the ileostomy and the inner ring 12 is pushed from the introducer 35, for instance using a simple pusher device, such as a plunger, which can provide stability during the delivery with the introducer. In the event a flexible endoscope is used, a flexible biopsy forceps can be inserted through the biopsy channel of the endoscope and detachably attached to the “pull-string” mechanism of the inner ring. When the inner ring 12 is sufficiently inserted into the lumen of the bowel, traction is applied to the biopsy forceps, which applies traction to the “pull string” device, which in turn deploys the inner ring 12. The “pull string” device is then released from the biopsy forceps, and the endoscope is withdrawn. The inner ring 12 resumes its original circular form and is deployed proximal to the sub-fascial space and is pulled into the sub fascial space where it lodges.

An embodiment with an inflatable inner ring 101 (uninflated ring 101′) is shown in FIG. 8B. In an uninflated state, the inner ring 101′ can be readily inserted into position. Once in position, the ring 101′ can be inflated and gently pulled into position. A balloon lumen can be used to supply fluid, such as air or saline, to the balloon. A fitting, such as a Luer lock fitting at the end of the balloon lumen can be used to connect to a syringe or other device to effectuate the fluid delivery or removal.

Referring to FIG. 9, in an alternative configuration, the device may be introduced into the bowel by a simple rod-shaped device, with a “V” shaped tip 38, inserted into the inner aspect of the sleeve 11, to engage the inner ring 12 or rings. The inner ring 12 or rings are placed under traction using the V tipped device 38 which deforms the inner ring or rings 12 into an elliptical shape, which facilitates their placement into the stoma without trauma or pressure, and then released, by removing the inserting device. This allows the inner ring 12 or rings to revert to its (their) original circular form and become lodged in the sub-fascial space. In an alternative configuration, the inner ring 12 may be introduced by applying traction to a pull string (not shown) of the inner ring 12 by a grasping device of a conventional upper endoscope or flexible sigmoidoscope, passed into the outer ring 13 and sleeve 11. In this configuration, the pull string attached to the inner ring 12 is grasped by a grasping device, such as a biopsy forceps, of a conventional endoscope. The biopsy forceps grasps the “pull string” and “pulls’ the inner ring 12 into the lumen of the stoma. Having reached the sub-fascial space, the “pull sting” is released and the inner ring 12 deploys in the lumen of the bowel, as described above. Similarly, the forceps can be used to directly hold the inner ring 12 in a suitable configuration to enter the stoma.

Referring to FIG. 10, the inner ring 12 is inserted into the lumen of the bowel, and after it resumes its previous circular form, it can be gently pulled up into the subfascial space 10 where it lodges because of the more narrowed circumference of the bowel at that point. Having removed the introducer, the outer ring 13 can then be rolled upon itself or otherwise pulled down until it reaches the skin of the anterior abdominal wall 6 and covers or protects the external stoma 36. A colostomy bag may be detachably attached to the outer ring 13 and prevents the need for glue or adhesives. Alternatively, the occlusive plates or cover 147 may be detachably attached to the stoma 36 and or the outer ring 13. Such an occlusive plate/cover 147 can be flat or bowl shaped to cover the stoma 36 and be secured to an appropriate securing structure. Cover 147 can act as a seal element with respect to discharge from the stoma. In one embodiment, the seal element can be any device used to close a gap fluid-tight with respect to liquids, gases, or both, such as a cap. Alternatively irrigating devices may be passed through the occluding plate to access the stoma or to extend into the stoma itself.

Referring to FIG. 11, there may be a plurality of inner rings 12, to increase traction within the bowel 34, and reduce chances of pressure necrosis of the bowel. These rings 12 may be equal to each other in diameter, in the manner of a concertina. The length of the membrane 11 between the rings 12 may be about 0.1-10 cm, in some embodiments about 0.2-5 cm, and in further embodiments about 0.5-2 cm. Referring to FIG. 12, in a further configuration, the diameter of the inner rings 12 may decrease progressively toward the sub-fascial space 10. In this configuration peristalsis of the bowel tends to compress the rings together, forming a pyramidal or conical form, so maintaining the inner rings 12 with more stability within the lumen. The ranges of lengths between rings also apply to the embodiment of FIG. 12. The ranges of diameters of the inner rings described above apply equally for the embodiments of FIGS. 11 and 12. A person of ordinary skill in the art will recognize that additional ranges of length within the explicit ranges above are contemplated and are within the present disclosure.

In yet another configuration, the device may be released from the bowel by means of a “pull string”, which is attached to the most proximal flexible ring of the device and deployed within the inner sleeve of the device. The “pull string” is of sufficient length that it protrudes from the ostomy and is easily identifiable and accessible to the operator. Referring to FIG. 13, the device has an outer ring 113, inner ring 112 which are interconnected by membrane 111, and a tether comprising a cord or “pull-string” 115 is attached to inner ring 111. For convenience, the terms cord, and pull-string are used interchangeably herein, and tether can refer to the cord/pull-string or the cord/pull-string along with the knob, as will be clear in the context. The free end of cord 115 can have a knob 114 to allow for easier gripping of the tether. As shown in FIG. 13, cord 115 is attached to inner ring 112 in a configuration that positions cord 115 to have a natural position on the exterior of the device. Referring to FIG. 14, the device is similar to the device of FIG. 13, except that cord 115 is attached to inner ring 112 at a position that has a natural position with cord 115 along the interior of membrane 111. If cord 115 is long enough, in either embodiment of FIG. 13 or FIG. 14, tether can be wrapped around inner ring 112 to place cord 115 in the interior of membrane 111 or along the outside of membrane 111. In general, cord 115 can be at least about 2 cm longer than membrane 111 to allow for use of the tether, in further embodiments at least about 5 cm longer, and in other embodiments from about 6 cm to about 30 cm longer than membrane 111, although there is no limit to the length other than practical convenience. A person or ordinary skill in the art will recognize that additional ranges of length within the explicit ranges above are contemplated and are within the present disclosure.

Gentle traction on the “pull string”, deforms the inner ring, and makes the diameter of the smaller dimension of the ring smaller, so making it simpler to pull through the stoma, and removing the entire device from the stoma, without trauma or pressure on the bowel. If the “pull string” or tether is deployed within the sleeve membrane, this ensures the inner ring is released and “pulled through” the inner lumen of the device, so potentially causing less pain to the patient. If the tether is located outside the inner and outer rings and the membrane, the tether would not interact with feces so the tether can remain cleaner, which would provide an advantage for this configuration. Referring to FIG. 15, engagement of cord 115 positioned outside of membrane 111 is used to disengage inner ring 112 from stoma 117 extending from abdominal wall 116. Referring to FIG. 16, removal of the device is shown using a pull string 115 passing through the interior of the membrane 111.

In a further embodiment, the rings may be expandable, using an inner spring located within the hollow lumen of the inner ring, which allows the ring to expand and contract. In another configuration, the inner ring may be expandable wherein the ring is discontinuous or cut in a radial direction. One end of the cut ring is thinner and can be inserted into the wider end so expandable, in a similar manner as a snake swallowing its own tail. With respect to the inflatable embodiment of FIG. 8B, deflation of inner ring 101 allows for easy removal of the device by pulling on outer ring 13 or a tether.

The device is therefore a self-retaining endoluminal device which avoids the need for conventional stoma plates and glues. The outer ring makes a stable platform where colostomy bags or occlusive plates or irrigation devices may be secured. This avoids the need for adhesive plates, glues, and powders. Since the lumen is an endoluminal device, no external plate is required and excoriation, inflammation or ulceration of the skin around the stoma is avoided.

Referring to FIG. 17, an embodiment of a colostomy bag 51 is shown having a fastener 52 for attachment to an outer ring of the ostomy device described herein. Fasteners 52 can have an appropriate design for secure attachment to the outer ring. Colostomy bag 51 can have a drainable design or be close with an intention of replacement when sufficiently full. Drainable ostomy pouches are described for example in U.S. Pat. No. 9,011,395 to Friske et al., entitled “Drainable Ostomy Pouch,” and in published U.S. patent application 2017/0209297 to Lysgaard, entitled “A Drainable Collection Bag,” both of which are incorporated herein by reference.

Referring to the insert of FIG. 17, the ostomy device can comprise a wafer-type structure 120 having a flange 121 for a snap fit to a colostomy bag 51 and fasteners 122 for attachment to an outer ring or as an alternative to the outer ring. The left view of the insert depicts the outer surface of wafer-type structure 120 and the right view depicts the inner surface of wafer-type structure 120. Having a wafer-type structure attached to the ostomy device still avoids the securing of a wafer to the patient's abdomen using adhesives, but can provide more familiar faster designs for securing an ostomy bag. Also, such a wafer can distribute some weight from the bag to the patient's abdomen without the need for adhesives and while avoiding irritation to the patient's skin due to possible contact with stool.

Referring to FIG. 18A, in a further embodiment, an inflatable balloon is 59 provided, attached to a fluid conduit 60, which is inflated in a similar manner as the balloon on a Foley catheter. The deflated balloon 59 is inserted through the lumen 61 of the deployed device 62 and positioned further within the patient's abdomen past the inner ring 13 of the deployed device. The balloon 59 is then inflated with fluid via the fluid conduit 60, as depicted in FIG. 18B. The fluid conduit has a valve 65 such as a Luer Lock fitting which maintains pressure within the balloon 59 and prevents leakage from the balloon 59.

Referring to FIG. 18B, the inflated balloon 59 is pulled gently toward the inner ring 13 of the device and a seal is formed between the inflated balloon 59 and the inner ring 12 of the device, similar to a “Ball valve” device. The inflated balloon 59 prevents stool, liquid and gas from exiting through the device and creates a stable mechanical seal between the balloon 59 and the inner ring 12.

The fluid conduit 60 may be secured at the outer ring 13 or a plate or the like 67 attached to the outer ring by a slot or key-hole type configuration which secures the balloon at the inner ring 12 and maintains the seal between the balloon and the inner ring.

An occluding plate/outer cover or cap which is attached to the outer ring may take several forms. In one embodiment shown in FIG. 19A, the outer cover 15 is a circular “clip-on” device which is detachably attached to outer ring 13 using clips or snaps 72. In a further embodiment, outer cover 15 performs as an occluding member and comprises a hinged disc or plate 73, with is detachably or non-detachably affixed to outer ring 13 at hinge 74. The terms plate, outer cover or cap can be used essentially interchangeably unless some specific meaning follows from the context. The hinged member can have a circular shape, with hinge 74 located at the circumference of the plate 73, and can be opened and closed, like a “port-hole” in a ship. In a further embodiment, the occluding member, such as outer cover 15, comprises a valve 75, which can have a plurality of “leafs”, as shown in FIG. 19A. The valve prevents stool gas and liquid from leaking from the device but allows the patient to insert an object such as an irrigating tube into the stoma, to irrigate the colostomy. An irrigating tube 77 inserted through valve 75 is shown in FIG. 19C. The valve may have 2 leafs, such as the mitral valve in the heart, or three leafs, such as the tricuspid valve in the heart, or a plurality of leafs. Outer cover 15 can also have a vent 76 in some embodiments. Vent 76 (FIG. 19A) can be open for passage of gas, and/or a releasable cover for selective release of gas, and vent 76 may have a filter, such as a charcoal filter, through which the gas can pass. Outer cover 15 may also have trapdoor 26 with a flap valve on the inner aspect of the trapdoor 26 to prevent leakage of stool or effluent (FIG. 19D). When it's desired to drain stool or effluent a drainage tube 28 can be inserted through the trapdoor 26 (FIG. 19E).

In a further embodiment, the occluding device incorporates a “gel” disc, similar to the commercially available “Gel Port” device (Applied Medical) used for laparoscopic abdominal surgery, and such gel disc products have been well described in the literature, see U.S. Pat. No. 7,736,306 and US 2020/0008792 to Becerra et al., entitled “Mechanical Gel Surgical Access Device,” both of which are incorporated herein by reference. The gel is a viscous, deformable jelly material which maintains its shape even when an object is inserted through the gel. Insertion is facilitated by a “cross-hairs” incision incorporated in the center of the gel. In this embodiment, a gel disc is detachably attached to the outer ring of the stoma device and can be used to access the lumen of the colon, via the device. The gel disc prevents leakage of stool liquid of gas from the colostomy but permits the insertion of objects such as drainage tubes. Such drainage tubes can be used to irrigate the colon, to empty it, or to drain accumulated fluids, such as in an ileostomy. The gel disc avoids the need to detach and reattach other occluding devices such as discs, plates, hinges plates, snap-on discs, clamp-on discs, ball valve devices, or valve devices.

In a further embodiment, the patient may wish to personalize the surface of the occluding member. Options may include a skin colored surface to match the patient's skin color. Other possibilities include light-hearted shapes or art such as hearts, cartoon characters, or “no entry” signs.

FIG. 20A illustrates the device with an inner colostomy bag 123 detachably attached to the outer ring 13 via an attachment mechanism, such as a snap-on or slip-on mechanism. The inner colostomy bag 123 has a top end and a bottom end and is configured to receive waste from the stoma. In this embodiment, the inner colostomy bag 123 includes an access hole at the bottom end, and waste passing through the access hole goes into the inner colostomy bag 123. Accordingly, as waste is expelled from the stoma and passes through the device, the waste is accumulated in the inner colostomy bag 123 rather than exiting the outer ring 13. The inner colostomy bag 123 may be constructed from latex, or a non-latex material such as plastic or a similar liquid impermeable material. The inner colostomy bag 123 may be transparent, translucent or opaque. In other embodiments, the inner colostomy bag 123 may be biodegradable.

In one embodiment, the inner colostomy bag comprises an elastic polymeric membrane having an elongation of at least 100% without breaking (i.e., an elongation at break of at least 100%). As used herein, “elongation at break” refers to the elongation recorded at the moment of rupture of the specimen, often expressed as a percentage of the original length; it corresponds to the breaking or maximum load, as measured by, for example, ASTM D-412 or ISO 37 and expressed as a percentage (%). In some embodiments, the elastic polymeric membrane has an elongation at break of at least about 150%, or at least about 200%, or at least about 250%, or at least about 300%, or at least about 350%, or at least about 400%, or at least about 450% or at least about 500%. In still other embodiments, the elastic polymeric membrane has an elongation at break of at least about 525%, or at least about 550%, or at least about 575%, or at least about 600%, or at least about 625%, or at least about 650%, or at least about 675%, or at least about 700%, or at least about 725%, or at least about 750%, or at least about 775%, or at least about 800%.

In another embodiment, the elastic polymeric membrane has a wall thickness of about 0.008 inches to about 0.025 inches, or about 0.010 inches to about 0.022 inches, or about 0.012 inches to about 0.020 inches, or about 0.014 inches to about 0.018 inches. A person of ordinary skill in the art will recognize that additional ranges of elongation and wall thickness within the explicit ranges above are contemplated and are within the present disclosure.

In one embodiment, the elastic polymeric membrane comprises a latex material, a natural rubbers, a thermoplastic polyolefin elastomer, a thermoplastic vulcanate, a thermoplastic polyurethane, a thermoplastic polyester, a thermoplastic co-polyester, a thermoplastic polyamide, a thermoplastic co-polyamide or a styrene block copolymer. Exemplary polymeric membranes include, but are not limited, to synthetic polyisoprene, butyl rubbers, polybutadiene, styrene-butadiene rubbers, chloroprene rubbers, polyacrylic rubbers, silicon rubbers, fluorosilicone rubbers, and nitrile rubbers such as Buna-N, hydrogenated nitrile rubbers, and nitrile butadiene rubber (NBR);
polypropylenes; polyurethanes; polyolefin elastomers, such as copolymers of ethylene, butane, and 1 or 2 octene; copolymers of ethylene and trans 2-butene; syndiotactic polyethylene;
isotactic polyethylene; water borne acrylics; latexes; and thermoplastic compounds, including thermoplastic polyoctene compounded with talc or titanium dioxide, thermoplastic elastomers compounded with thermoplastic polymers, thermoplastic polyurethane elastomers and thermoplastic elastomers (TPE) alone or compounded with thermoset polymers.

FIGS. 20B-20D illustrate a waste collection system being assembled and disassembled for the collection and disposal of waste from the colon or ilium of a patient that would otherwise move through a stoma according to an exemplary embodiment of the present subject matter. As shown, waste collection system comprises the endoluminal stoma channel device 200 of the present disclosure, an inner colostomy bag 123 and outer cover 15 which are described in more detail above. In FIG. 20B, the inner colostomy bag 123 is detachably attached to the outer ring 13 with the top end of the inner colostomy bag 123 gently forced through the access hole at its bottom end and outer ring 13 so that the top end is positioned within the inner aspect 124 of the sleeve of the device. The outer cover 15 can then be detachably attached to the outer ring 13 to secure the inner colostomy bag 123 within the device. In FIG. 20C, when it's desired to expel waste from the stoma, the outer cover 15 can be detached from outer ring 13. The flow of waste 125 will force the top end of the inner colostomy bag 123 back through the access hole/outer ring 13 so that the top end of the inner colostomy bag 123 is positioned outside of the device. When the inner colostomy bag 123 has been in use for some time and is at least partially filled with waste 125, the inner colostomy bag 123 can be detached from the outer ring 13 and emptied (FIGS. 20C and 20D). If desired, the user may squeeze the remaining contents out of the inner colostomy bag 123 by stroking or massaging movements in the direction towards the bottom end/access hole of the inner colostomy bag 123. When emptied, the bottom end/access hole of the inner colostomy bag 123 may be thoroughly rinsed. If desired, the inner colostomy bag 123 (or a new inner colostomy bag 123) may then be releasably attached to device for use in collecting additional waste.

With reference now to FIG. 21, in another embodiment the present disclosure provides a double-barreled ostomy device 129 for loop colostomies and ileostomies. In this embodiment, the device has two separate limbs 130, 131 one for each loop of the stoma. The inner rings 132, 133 of the two lumen device may be inserted in the afferent and efferent limb of the stoma. The two lumens communicate with a common outer ring 134 which can be folded to the abdominal wall in the same manner as the single limb stoma. The device, inner rings 132, 133 and outer ring 134 may be made from the same materials and function similarly to those described above.

Referring now to FIGS. 22A and 22B, in another embodiment the inner colostomy bag 123 includes a malleable compressible colostomy ring 126 disposed around the access hole of the inner colostomy bag 123. The colostomy ring 126 may be compressed and inserted below the outer ring 13 and after compression the colostomy ring 126 is configured to return to or close to its original shape to sealingly engage the outer ring 13 and prevent leakage of waste or gas from the stoma. The colostomy ring 126 may be made of either flexible, soft or semi-rigid polymer or plastic, spring metal (e.g., Nitinol) or combinations thereof.

In another embodiment, the colostomy ring 126 may further include a tab 127 or string attached to the periphery of the colostomy ring 126. The tab 127 may have an essentially flat shape and extends between a first end which is secured to the ring 126 and a second end which is free and can be grasped by gripping so as to positively move the ring end part on which the tab 127 is mounted. In the event of gas build-up, the tab 127 may be used to release gas from the lumen of the bowel by gripping and applying traction to the tab 127 causing the colostomy ring 26 to deform thereby breaking the seal between the colostomy ring 126 and the outer ring 13 and allowing the gas or flatus to be released. Once the gas has been released, the colostomy ring 126 can be repositioned 28 below the outer ring 13 to sealingly engage the outer ring 13 by applying pressure to the tab 127 or string. Ideally, the tab 127 or string may be located at a 12 o'clock position on the colostomy ring 126 to preferentially allow the escape of gas or flatus located above the fluids or stool, rather than fluids or stool themselves.

In a further embodiment, the tab 127 or string may be detachably attached to the periphery of the outer cap 15, and partial removal of the outer cap 15 will result in the application of traction to the tab 127 or string causing the colostomy ring 126 to deform thereby breaking the seal between the colostomy ring 126 and outer ring 13 resulting in the release of gas or flatus. In still a further embodiment the tab 127 or string may protrude through an opening on the outer cap 15 allowing the user to grip and apply gentle traction on the tab 127 or string causing the colostomy ring 126 to deform the inner ring thereby breaking the seal between the outer ring 13 and colostomy ring 126 resulting in the release of gas or flatus, without removal of the outer cap 15 itself. The colostomy ring 126 may be returned to the previous sealingly engaged position by gripping and applying pressure to the tab 127 or string. These configurations are preferably located at the 12 o'clock position on the colostomy ring and cap, to preferentially allow the escape of gas, and not liquid

In still another embodiment, the inner colostomy bag 123 may further include a gas release mechanism (not shown). For example, the gas release mechanism may include an air release tube having an air release opening on its upper end an air intake opening on its lower end and is sized and configured to be received within a port on the inner colostomy bag 123 such that the air release opening is external to the inner colostomy bag 123 and the air intake opening is inside the inner colostomy bag 123. As those of skill in the art will recognize, gas is periodically expelled from the stoma and may cause colostomy bags to quickly fill with gas, rather than solid waste. In order to reduce the difficulty involved with releasing gas from the inner colostomy bag 123, embodiments are described, for example, of a gas release mechanism having an air release tube inserted into a port on the inner colostomy bag such that gas inside the colostomy bag 123 passes into the air intake opening and exits the air release opening.

In one embodiment, the air release tube may comprise a threaded portion that is configured to threadedly engage the port on the inner colostomy bag 123. The air release tube may be inserted into the inner colostomy bag by rotating the air release tube while applying pressure between the threaded portion and the correspondingly sized port in the inner colostomy bag 123.

The air release tube may comprise any type of tubing, such as flexible rubber tubing, plastic tubing, or metal tubing, and is configured for insertion into the inner colostomy bag 123 via the port. When the patient, or caregiver to the patient, desires to release gas from the inner colostomy bag 123, the air release tube may be inserted into the inner colostomy bag via the port, and when pressure is applied to the inner colostomy bag 123, gas within the inner colostomy bag 123 will exit the air release tube. Accordingly, the inner colostomy bag 123 may be used for an extended period of time due to the evacuation of gas that periodically accumulates within the inner colostomy bag 123.

In one embodiment, the air release tube inserted into the inner colostomy bag may comprise a mechanism for releasably blocking the air release opening in the tube. For example, the air release tube comprises a blocking mechanism that may be used to block the opening of the air release tube in order to seal the contents of the inner bag colostomy bag 123. In order to reduce the risk of waste from the inner colostomy bag 123 escaping inadvertently through the air release tube, the blocking mechanism, when inserted into the air release opening of the air release tube, seals the air release tube thereby preventing passage of waste out of the air release opening. Other mechanisms for sealing an end of the air release tube, including valves, are also contemplated and are usable within the scope of the devices and methods described herein.

In another embodiment, the air release tube also comprises a threaded portion that provides a coupling between the air release tube and the port. In this embodiment, the air release tube may be inserted into the inner colostomy bag by rotating the air release tube while applying pressure between the threaded portion and the hole. The spiral threads on the threaded portion cause the air release tube to enter the inner colostomy bag through the port. Advantageously, the threaded portion provides an enhanced engagement of the air release tube and the inner colostomy bag. In other embodiments, other attachment means, such as protrusions or indentations on the air release tube, may be used in order to engage the air release tube to the inner colostomy bag 123.

In practice, the ostomy device can be used for an extended period of time or replaced at a recommended period, such as daily, weekly, monthly or other appropriate period. To this end, the device can be designed to be inserted and removed by a physician, an ostomy nurse, other health care profession or the patient themselves. Generally, the ostomy bag is emptied and/or replaced by the patient or a care provider on a time frame generally of multiple times a day on average.

### Long Term Use/Biocompatible Material

Commercial devices such as the “Alexis” retractor is designed for short term use (typically 1-2 hours) as an abdominal wall retractor during surgical laparotomy, or as a skin retractor, as during breast biopsies. The membrane material is designed for contact with the patient's tissue for just the duration of the surgical procedure, for instance several hours, but no longer than 24 hours.

In the current devices, components of the device can remain in place for a much longer period of time, and the membrane and ring material can be biocompatible with the patient's tissues for days, or weeks. A patient with a prior art colostomy typically changes the palate 2 or 3 times a week, to avoid skin irritation, or fungal or bacterial overgrowth. With the current system, the device may be changed every week or two, or only when problems arise, such as dislodgement of the device or leakage from the device. This longer term contact with the patient's tissues can suggest the selection of a longer-term biocompatible material for the rings and sleeve components. Suitable polymers include, for example, polyamides (e.g., nylon), polyesters (e.g., polyethylene teraphthalate), polyacetals/polyketals, polyimide, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, natural and synthetic rubbers, similar copolymers and mixtures thereof. Suitable metals can include, for example, titanium, stainless steel, and alloys such as Nitinol and other spring metals.

Often patients with colostomies irrigate the stoma once a day, to empty the colon of liquid, gas and stool. After irrigating, these patients typically apply a large “band-aid” device over the stoma, to conceal the stoma, and minimize leakage. The current device allows for irrigation of the colon on a daily basis, and the occluding device (Plate, trapdoor, cap, gel-cap, valve or ball valve) is reapplied to prevent leakage.

Commercial retractors such as the Alexis retractor are designed to provide access to the abdominal cavity, Uterus (in Ceserian Sections) or skin, such as in breast biopsies, but these devices are not designed for longer term use or secure attachment to the patient away from supervision by a health care professional. The current system and associated device components has a plurality of designs and functions. A major function is to occlude a stoma and prevent leakage of stool gas and liquid. Various mechanisms are described herein which accomplish this: Using the ball-valve, hinged-plate, snap-on, a simple cap, screw on, gel-cap, or valve occluding devices. This function is distinct from vaguely similar commercial devices, which are designed to open access into a body cavity. A further function is to create a stable platform to attach colostomy bags, and to avoid the use of prior art colostomy plates and adhesives.

### Patient Experience

A 57 year old morbidly obese male underwent a “Hartman's resection” for perforated diverticulitis. After multiple post-operative complications including sepsis, respiratory and cardiac failure, he survived but was hospitalized for over a month. Because of his obesity, and a 5 in deep abdominal wall, the end of the colostomy underwent ischemic necrosis, leaving a pencil thin “blow-hole+ fistula (FIG. 20). This resulted in severe back-up of stool, causing severe abdominal pain, and persistent problems “pouching” the stoma, due to the patient's body habitus. The device was inserted into the “blow-hole” colostomy and deployed in the stoma, forming a stable platform for placement of a colostomy bag (FIG. 21). The device remained in place and even applying significant traction to the device failed to dislodge the device (FIG. 22). The patient underwent a successful bowel prep to clean out the colon, and had surgery to reverse the colostomy, and re-anastomose the colon to the rectum.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated.

1. A waste collection system for collecting waste from a body of a patient, the waste collection system comprising:
an endoluminal stoma channel comprising a resilient deformable inner ring, an outer ring, a flexible polymer sleeve comprising a biocompatible polymer and interconnecting the inner ring and the outer ring;
an inner colostomy bag configured to be detachably attached to the outer ring, wherein the inner colostomy bag comprises an elastic polymer membrane with an elongation of at least about 100% without breaking; and
a cover comprising a clamp or seal element configured to engage the outer ring in a sealed engagement with the inner colostomy bag attached to the outer ring, wherein a portion of the flexible polymer sleeve and the inner ring are configured for insertion into a stoma formed in the body of the patient.

2. The waste collection system of claim 1 wherein the inner colostomy bag is biodegradable.

3. The waste collection system of claim 1 wherein the inner colostomy bag comprises latex.

4. The waste collection system of claim 1 wherein the inner colostomy bag comprises a colostomy ring positioned at a mouth of the inner colostomy bag and configured to sealingly engage the outer ring of the endoluminal stoma channel, wherein the colostomy ring is resilient and deformable.

5. The waste collection system of claim 1 wherein the colostomy ring comprises a tab positioned on the periphery of the colostomy ring.

6. The waste collection system of claim 1 wherein the outer ring comprises a non-circular cross section that can be inverted to fold over the flexible polymer sleeve.

7. The waste collection system of claim 1 wherein the inner ring comprises circumferential protrusions.

8. The waste collection system of claim 1 wherein the flexible polymer sleeve is transparent.

9. The waste collection system of claim 1 wherein the inner colostomy bag is detachably attached to the outer ring by a snap-on mechanism or a slip-on mechanism.

10. The waste collection system of claim 1 wherein the inner colostomy bag and/or the cover further comprises a gas releasing mechanism configured to be detachably attached to inner colostomy bag.

11. The waste collection system of claim 10 wherein the gas releasing mechanism comprises an air release tube.

12. The waste collection system of claim 1 further comprising an introducer configured to insert the inner ring and portion of the flexible polymer sleeve into the stoma.

13. The waste collection system of claim 12 wherein the introducer is a tubular introducer having a distal opening and a proximal opening configured to releasably hold a pusher device.

14. An endoluminal stoma channel comprising:
an inner ring that is resilient and deformable, wherein the inner ring in its undeformed configuration has an outer surface comprising contours and/or texture to present contact points of polymer having a shore durometer value of no more than 65 A;
an outer ring; and
a flexible polymer sleeve connected to the inner ring and the outer ring to form a flow channel and comprising a biocompatible polymer.

15. The endoluminal stoma channel of claim 14 wherein the outer ring comprises a polymer having a shore durometer value greater than 65 A.

16. The endoluminal stoma channel of claim 14 further comprising a tether comprising a cord and a knob, wherein cord is attached to the inner ring, and the knob has a position such that the cord can extend along the sleeve, in the interior or adjacent the exterior of the sleeve, past the outer ring with the outer ring pulled away from the inner ring such that the sleeve is taut.

17. The endoluminal stoma channel of claim 14 wherein the outer surface comprises contours having one or more concave, ogee, French curve, arch, or hook regions.

18. The endoluminal stoma channel of claim 14 wherein the outer surface is textured having one or more nubs, bumps, ribs, or protrusions.

19-20. (canceled)

21. A method for. collecting waste from a stoma through an endoluminal stoma channel comprising a resilient deformable inner ring, an outer ring, and a flexible polymer sleeve connected to the inner ring and the outer ring to form a flow channel and comprising a biocompatible polymer, the method comprising:
engaging an inner colostomy bag onto the outer ring and positioning the inner colostomy bag in the inner aspect of the flexible polymer sleeve wherein the inner colostomy bag comprises an elastic polymer membrane with an elongation of at least about 100% without breaking;
engaging a cover with a clamp onto the outer ring of the endoluminal stoma channel, wherein the inner ring is located within the patient with the flexible polymer sleeve extending outward from the stoma with the outer ring positioned outside the stoma, wherein the inner colostomy bag and cover provides closure of the stoma or a controlled pathway for releases from the stoma; and
disengaging the cover from the outer ring allowing waste from stoma to be collected within the inner colostomy bag.

22-23. (canceled)
